SEQUENCE LISTING (6 sequences)

SEQ ID NO: 1 - DNA, 3736 bp, Homo sapiens
  CDS (114)..(1223)
  sig_peptide (114)..(149)
ggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacggtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcggtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactactccaggagtaggaaatgattcatcatcctcccaaagcaagaggcttaagagagaaacaccgaaattcagatagctcagggactgctaacagagaactacatttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaaaaa

SEQ ID NO: 2 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 3 - DNA, 3718 bp, Homo sapiens
  CDS (114)..(1205)
  sig_peptide (114)..(149)
ggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacggtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcggtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactactccaggagtaggaaatgattcatcatcctcccaaagcaagaggcttaagagagaaacaccgaaattcagatagctcagggactgctaacagagaactacatttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaaaaa

SEQ ID NO: 4 - protein, 364 aa, Homo sapiens
MHRLIFVYTLICANFCSCRDTSATPQSASIKALRNANLRRDDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 5 - DNA, 1404 bp, Homo sapiens
  CDS (402)..(821)
  sig_peptide (402)..(437)
cgccgccgctctgagcccgagtgcgcgcctctcaggggccgcggccggggctggagaacgctgctgctccgctcgcctgccccgctagattcggcgctgcccgccccctgcagcctgtgctgcagctgccggccaccggagggggcgaacaaacaaacgtcaacctgttgtttgtcccgtcaccatttatcagctcagcaccacaaggaagtgcggcacccacacgcgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaaaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacctttccagcaca

SEQ ID NO: 6 - protein, 140 aa, Homo sapiens
MHRLIFVYTLICANFCSCRDTSATPQSASIKALRNANLRRDDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRKPN

(19) United States
(12) Patent Application Publication
(10) Pub. No.: US 2003/0100502 A1
(43) Pub. Date: May 29, 2003

(54) Treating musculoskeletal disorders using lp85 and analogs thereof

(76) Inventors: John Michael Beals, Indianapolis, IN (US); Patricia Ann Gonzalez-DeWhitt, Noblesville, IN (US); Lisa Janine Hammond, Carmel, IN (US); Jirong Lu, Indianapolis, IN (US); Songqing Na, Carmel, IN (US); Eric Wen Su, Carmel, IN (US); Derrick Ryan Witcher, Fishers, IN (US)

Correspondence Address: ELI LILLY AND COMPANY, PATENT DIVISION, P.O. BOX 6288, INDIANAPOLIS, IN, 46206-6288, US

(21) Appl. No.: 10/258,557

(22) PCT Filed: May 8, 2001
(86) PCT No.: PCT/US01/11755

(51) Int. Cl. A61K 38/17; C07K 14/775; C12P 21/02; C12N 5/06; C07H 21/04

(52) U.S. Cl. 514/12; 435/69.1; 435/320.1; 435/325; 530/359; 536/23.5

(57) ABSTRACT

The invention provides methods for treating or preventing osteoporosis, osteopenia, sarcopenia, arthritis, tissue atrophy, wound healing, traumatized connective tissues, grafted connective tissues, and/or transplanted organs in a mammal which comprise the administration of a therapeutically effective amount of a pharmaceutical composition comprising a LP85 polypeptide or LP85 analog.

### CROSS-REFERENCE

This application claims the benefit of U.S. Provisional Application Nos. 60/205,424 filed May 19, 2000; No. 60/261,071 filed Jan. 11, 2001; and No. 60/261,076 filed Jan. 11, 2001.

### FIELD OF THE INVENTION

The present invention relates generally to recombinant DNA technology as applied to the field of human medicine. In particular, the invention relates therapeutic uses of LP85, a platelet-derived growth factor (PDGF) homolog containing an N-terminal CUB domain and a hinge region, and analogs thereof.

### BACKGROUND OF THE INVENTION

PDGF was initially described by Ross et al. as a factor found in whole blood serum that is capable of supporting the growth of fibroblasts in culture (Proc. Natl. Acad. Sci. USA, 71:1207-1210 (1974)). PDGF was subsequently isolated from platelets and from serum, with the native unreduced PDGF being identified as a 27-35-kDa dimeric protein. Reduction of PDGF yields two or more subunits in a molecular weight range of approximately 18 kDa and 16 kDa, respectively referred to as the “A” and “B” subunits. The A subunit is approximately 35% homologous to the B subunit. The PDGF B subunit from human platelets comprises a 111 amino acid cleavage product of a 241 amino acid precursor polypeptide (Johnsson et al, EMBO Journal, 3(5), 921-928 (1984)). PDGF is believed to be biologically active only in dimeric form. Biologically active PDGF dimers can take the form of a PDGF A-B heterodimer, a PDGF B-B homodimer, or a PDGF A-A homodimer (see, e.g. Hannink et al, Mol. Cell. Biol., 6, 1304-1314 (1986)). Each monomeric subunit of the biologically active dimer, irrespective of whether it is an A subunit or a B subunit, contains eight cysteine residues. Two of these cysteines form interchain disulfide bonds to hold the dimer together and the rest of the cysteine residues are involved in disulfide bonds in the cystine knot motif. The 111 amino acid sequence (PDGF B.sub.111) identified as the mature form of PDGF B, has been produced in yeast and other eukaryotic host cells.

Recently, much attention has been paid to the use of growth factors to accelerate wound healing, particularly of skin. The use of PDGF to accelerate wound healing in skin and connective tissue has been investigated (Antoniades et al., Proc. Natl. Acad. Sci. USA 88:565-569 (1991); Cromack et al., J. Trauma 30:S129-133 (1990); Ross et al., Philos. Trans. R. Soc. Lond. (Biol.) 327:155-169 (1990)). Human PDGF is believed to be the major mitogenic growth factor in serum for connective tissue. PDGF has been shown to induce mitogenesis in arterial smooth muscle cells, fibroblast cell lines, and glial cells (See e.g. Deuel et al, J. Biol. Chem., 256(17), 8896-8899 (1981); Heldin et al, J. Cell Physiol., 105, 235 (1980) (brain glial cells); Raines and Ross, J. Biol. Chem., 257, 5154 (1982) (monkey arterial smooth muscle cells)). PDGF is also believed to be a chemo-attractant for fibroblasts, smooth muscle cells, monocytes, and granulocytes. Because of its apparent abilities to induce mitogenesis and to attract fibroblasts to the site of wounds, PDGF could have therapeutic utility in the repair of injured or traumatized connective tissues.

A newly identified PDGF family member was described in international patent applications WO 00/27879 and WO 00/34474 and alternatively named PDGF-D and ZVEGF3, respectively (the entire contents of which are incorporated herein by reference). The PDGF-D protein is a member of the cysteine knot family, however, it is one of only three PDGF related proteins found to have a CUB domain. Another recently identified PDGF family member containing a CUB domain was identified from chick spinal cord tissue (Hamada T, Ui-Tei K, and Miyata Y., 2000, FEBS Letters, 475:97-102). Its expression was increased in chick spinal cords during embryonic development. The amino acid sequence of this protein revealed a CUB domain followed by a region homologous to the members of the PDGF family. This protein is thought to play an important role in the development of the spinal cord.

The CUB domain is a widespread structural motif found in functionally diverse proteins. Many of the proteins which possess CUB domains are known to be involved in the regulation of development [Bork P and Beckmann G., 1993, J. Mol. Bio., 231:539-545]. CUB domains contain approximately 110 amino acid residues and are named after the first three proteins in which it was identified (C1r/C1s complement subcomponents, Uegf embryonic sea urchin protein, BMP1 bone morphogenetic protein). Multiple copies of the CUB domain have been found in various proteins: two CUB domains in C1r/C1s, three CUB domains in BMP1, five CUB domains in TOLLOID (Drosophila dorso-ventral patterning gene product). A single CUB domain is found in spermadhesins, which are involved in sperm-egg binding. These proteins perform diverse functions: the CUB domain in C1r mediates its interaction with C1s in a calcium-dependent manner (Thielens NM et al., J. Bio. Chem., 274:9149, (1999)), while spermadhesin PSP-I/PSP-II CUB domain interacts with carbohydrates (Topfer-Petersen E et al., Andrologia, 30, 217, (1998)). However, relatively little is known about the role of CUB domains in these proteins.

Sarcopenia is a major determinant of age-related disabilities that is characterized by a decline in muscle mass, muscle weakness, and increased fatigability (See, e.g. P. Balagopal et.al., Endocrine, 7, 57-60, (1997); K. Short and K. Nair, J. Endocrinol. Invest., 22, 95-105, (1999)). These changes produce substantial physical disability in the elderly. The quality and quantity of muscle depends on the integrity of a continuous remodeling process that includes breakdown of old proteins and synthesis of new ones. The maintenance of muscle is determined by a delicate balance between these two processes, implying that a decline in muscle mass occurs when protein breakdown exceeds synthesis. The quality of life of individuals suffering from sarcopenia and other related musculoskeletal disorders would be improved by a compound that impeded or reversed the degenerative process of these conditions.

As knowledge about bone growth and strength has progressed over the years, one approach to treat or prevent reduction in bone mass has involved the use of therapeutic peptides or polypeptides (for review, see international patent application WO 94/20615 published on Sep. 15, 1994, the contents of which are incorporated herein by reference).

In view of the well-recognized utility of peptides and polypeptides in treating various tissue growth disorders, there is a need in the art to identify and provide molecules that are useful therapeutic agents. Accordingly, it is an object of the present invention to provide for the treatment of conditions that admit to the use of native PDGF-D and PDGF-D analogs having longer circulatory half-lives and slower clearance rates from plasma relative to natural PDGF-D. It is also an object of this invention to provide PDGF-D molecules that exhibit a longer half-life and slower clearance rate from plasma relative to that of native PDGF-D molecules.

### SUMMARY OF THE INVENTION

Applicants have identified structural modifications to native PDGF-D, hereinafter referred to as LP85, polypeptides that enhance the biological significance of the CUB domain structure in LP85 polypeptides. The modifications disclosed herein result in LP85 analogs with enhanced structural stability in vitro and in vivo, that results in greater stability and higher tissue specificity in vivo.

The present invention provides novel LP85 analogs that are modified from the native form such that the molecules are pharmaceutically more desirable than the native forms of LP85 polypeptide. In one embodiment, the present invention relates to a pharmaceutical composition comprising LP85 or an LP85 analog, together with one or more pharmaceutically acceptable diluents, carriers, or excipients.

Another object of the present invention is to provides novel methods of treating musculoskeletal disorders in a mammal that comprise administering to said mammal a therapeutically effective amount of a pharmaceutical composition comprising at least one LP85 polypeptide or analog thereof. The compositions of the present invention are particularly useful for treatment or prevention of musculoskeletal disorders including, but not limited to, osteoporosis, osteopenia, sarcopenia, various forms of arthritis, tissue atrophy, peridontal disease, wound healing, traumatized connective tissues, grafted connective tissues and/or transplanted organs, or bone or muscle loss due to malignancy, endocrine disorders, arthritis, immobility, or disuse.

The invention also provides compositions for and methods of prophylactically increasing or maintaining bone density and/or bone quality in a subject having a substantially normal bone density. Such methods comprise the step of administering to a mammal a biologically?? Defined?? effective amount of a pharmaceutical composition comprising at least one LP85 polypeptide or analog thereof.

### BRIEF DESCRIPTION OF THE DRAWINGS

FIG. 1 is a graph that illustrates LP85 stimulation of ³H-thymidine uptake in BalbC/3T3 Fibroblasts as compared to the inactivity of cleavage-resistant mutants.

FIG. 2 shows LP85 induction of Map Kinase and Tyrosine Phosphorylation in BalbC/3T3 Fibroblasts. Further studies revealed that protein preparations containing C-terminal cleavage products were active, while those containing pure full-length protein were not. Cleavage resistant mutants had no activity in this assay.

FIG. 3 is a graph that illustrates LP85 stimulation of ³H-thymidine uptake in human dermal fibroblasts.

FIG. 4 is a graph that illustrates LP85 stimulation of ³H-thymidine uptake in rat L6 skeletal muscle cells.

FIG. 5 is a graph showing that LP85-induced proliferation of BalbC/3T3 fibroblasts is blocked by soluble PDGF Receptor-S.

FIG. 6 indicates that LP85 stimulates tyrosine phosphorylation of the PDGFR-β in BalbC/3T3 fibroblasts.

FIG. 7 is a graph that indicates LP85 Stimulation of Rat Osteosarcoma cells weakly induces osteocalcin promoter.

FIG. 8 is a graph illustrating anabolic bone activity of LP85 in a Rat neonate metatarsal model.

FIG. 9 is a table data showing that LP85 partially reverses IL-1⊖-induced reduction of proteoglycan synthesis.

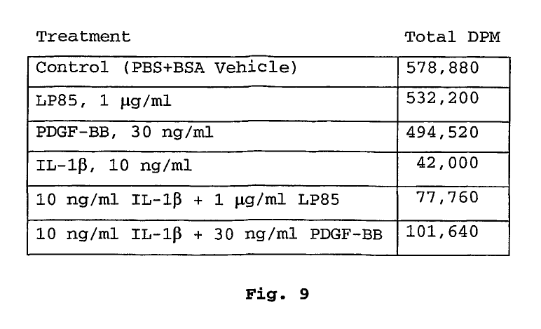

FIG. 10 is a graph illustrating LP85 Stimulates chemotaxis of human dermal fibroblasts.

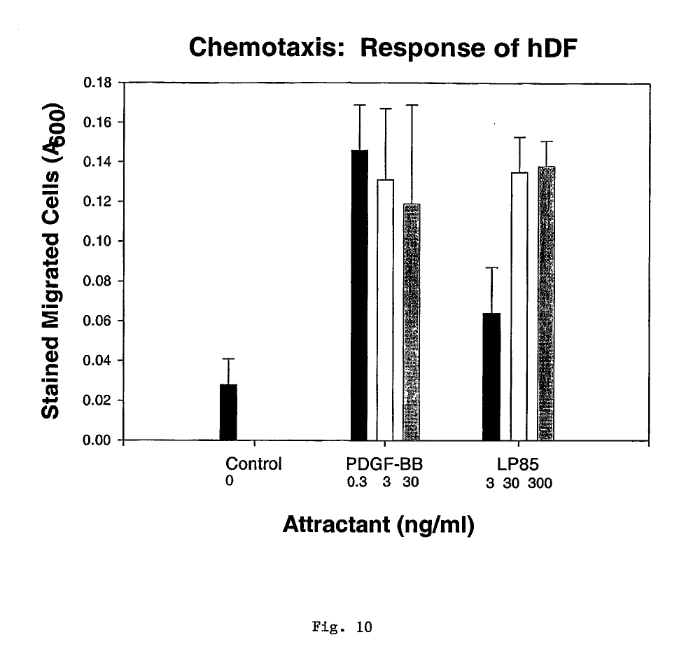

### DETAILED DESCRIPTION OF THE INVENTION

Definitions

The term “activity” or the phrase “biological activity” in reference to a LP85 polypeptide or LP85 analog relates to the capacity of the particular LP85 polypeptide or LP85 analog to induce, in vivo and/or in vitro, the biological consequences associated with such molecules by the present disclosure, including induction of mitogenic activity in endothelial cells, skeletal and smooth muscle cells, fibroblast cells, osteoblasts, and/or bone growth as well as inhibition of IL-1β-induced inhibition of proteoglycan synthesis. Accordingly, LP85 polypeptide or LP85 analog activity can be assessed by one or more of the in vitro or in vivo assays disclosed herein or otherwise known in the art.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids such as norleucine, β-alanine, ornithine, etc.; and chemically synthesized compounds having properties known in the art to be characteristic of amino acids.

The incorporation of non-natural amino acids, including synthetic non-native amino acids, substituted amino acids, or one or more D-amino acids into the LP85 analogs of the present invention (“D-LP polypeptides”) is advantageous in a number of different ways. D-amino acid-containing polypeptides exhibit increased stability in vitro or in vivo compared to L-amino acid-containing counterparts. Thus, the construction of polypeptides incorporating D-amino acids can be particularly useful when greater stability is desired or required in vivo. More specifically, D-peptides are resistant to endogenous peptidases and proteases, thereby providing improved bioavailability of the molecule, and prolonged lifetimes in vivo when such properties are desirable. When it is desirable to allow the peptide to remain active for only a short period of time, the use of L-amino acids therein will permit endogenous peptidases, proteases to digest the molecule, thereby limiting the cell's exposure to the molecule. Additionally, D-peptides cannot be processed efficienty for major histocompatibility complex class II-restricted presentation to T helper cells, and are therefore less likely to induce humoral immune responses in the whole organism.

In addition to using D-amino acids, those of ordinary skill in the art are aware that modifications in the amino acid sequence of a peptide, polypeptide, or protein can result in equivalent, or possibly improved, second generation peptides that display equivalent or superior functional characteristics when compared to the original amino acid sequences. Alterations in the LP85 analogs of the present invention can include one or more amino acid insertions, deletions, substitutions, truncations, fusions, shuffling of subunit sequences, and the like, either from natural mutations or human manipulation, provided that the sequences produced by such modifications have substantially the same (or improved or reduced, as may be desirable) activity(ies) as the LP85 analog sequences disclosed herein. The term “LP85 analog” refers to any modified form of a LP85 polypeptide that exhibits substantially the same or enhanced biological activity in vivo and/or in vitro as compared to the corresponding unmodified form and is pharmaceutically more desirable, in at least one aspect, as compared to the corresponding unmodified LP85 polypeptide. As used herein, the term “LP85 analog” is intended to encompass LP85 polypeptides as defined herein wherein the LP85 polypeptide further comprises at least one modification not normally native to LP85 polypeptides. The term “modification” includes any change in structure (ie., a qualititive change) of a protein. Such modifications can include, but are not limited to, changes in the amino acid sequence, transcriptional or translational splice variation, pre- or post-translational modifications to the DNA or RNA sequence, addition of macromolecules or small molecules to the DNA, RNA or protein, such as peptides, ions, vitamins, atoms, sugar-containing molecules, lipid-containing molecules, small molecules and the like, as well-known in the art. One type of protein modification according to the present invention is by one or more changes in the amino acid sequence (substitution, deltion or insertion). Such changes could include, at one or more amino acids, a change from a charged amino acid to a different charged amino acid, a non-charged to a charged amino acid, a charged amino acid to a non-charged amino acid as discussed, infra. or supra. Any other change in amino acid sequence is also included in the invention. Another type of protein modification is by changes in processing of the protein in the cell. A non-limiting example is where some proteins have an “address label” specifying where in (or outside of) the cell they should be used. Such a label or tag can be in the form of a peptide, a sugar or a lipid, which when added or removed from the protein, determines where the protein is located in the cell. A further type of protein modification is due to the attachment of other macromolecules to a protein. This group can include, but is not limited to, any addition/removal of such a macromolecule. These molecules can be of many types and can be either permanent or temporary. Examples include: (i) polyribosylation, (ii) DNA/RNA (single or double stranded); (iii) lipids and phosphlipids (e.g., for membrane attachment); (iv) saccharides/polysaccharides; and (v) glycosylation (addition of different types of sugar and sialic acids—in a variety of single and branched structures). Another type of protein modification is due to the attachment of other small molecules to proteins. Examples can include, but are not limited to: (i) phosphorylation; (ii) acetylation; (iii) uridylation; (iv) adenylation; (v) methylation, and (vi) capping (diverse complex modification of the N-terminus of the protein for assorted reasons). Most of these changes are often used to regulate a protein's activity. (v) and (vi) are also used to change the half-life of the protein itself. These protein changes can be detected on 2 dimensional gel electrophoresis incorporating several methods, such as labeling, changes in pI, antibodies or other specific techniques directed to the molecules themselves, as known in the art. Molecular weight changes can be, but may not usually be detectable by 2DGE. MALD (matrix assisted laser desorption of flight mass spectrometry) is preferred to detect and characterize these modifications. Such modifications are generally directed at improving upon the poor therapeutic character of the native LP85 polypeptide by increasing that molecule's target specificity, solubility, stability, serum half-life, affinity for targeted receptors, susceptibility to proteolysis, resistance to clearing in vivo, ease of purification, and/or decreasing the antigenicity and/or required frequency of administration.

The terms “complementary” or “complementarity” as used herein refer to the capacity of purine and pyrimidine nucleotides to associate through hydrogen bonding to form double stranded nucleic acid molecules. The following base pairs are related by complementarity: guanine and cytosine; adenine and thymine; and adenine and uracil. As used herein, “complementary” means that the aforementioned relationship applies to substantially all base pairs comprising two single-stranded nucleic acid molecules over the entire length of said molecules. “Partially complementary” refers to the aforementioned relationship in which one of two single-stranded nucleic acid molecules is shorter in length than the other such that a portion of one of the molecules remains single-stranded.

The term “fragment thereof” in reference to a LP85 gene or cDNA sequence, refers to a fragment, or sub-region of an LP85 nucleic acid such that said fragment comprises 10 or more nucleotides that are contiguous in the native nucleic acid molecule as shown in SEQ ID NO:1.

The term “fragment thereof” in reference to a LP85 polypeptide or LP85 analog refers to a fragment, or sub-region, of an LP85 polypeptide or LP85 analog such that said fragment comprises 5 or more amino acids that are contiguous in the native LP85 polypeptide as shown in SEQ ID NO:2 or contiguous in the LP85 analog, as the case may be.

“Functional fragment,” as used herein, refers to an isolated sub-region, or fragment of a protein, or sequence of amino acids that, for example, comprises a functionally distinct region such as an active site on an enzyme, or a binding site for a ligand, receptor, polypeptide, or other substrate. Functional fragments may be produced by recombinant DNA methodologies, enzymatic/proteolytic digestions, or as natural products of alternative splicing processes.

“Functionally-related” as used herein is applied to proteins or peptides that are predicted to be functionally similar or identical to a particular protein or peptide. Molecules that would be expected to be functionally related to LP85 polypeptides are those that are sufficiently homologous in their amino acid composition as compared with LP85. For example, one or more conservative amino acid substitutions or deletions in the native LP85 polypeptide or in a LP85 analog of the present invention would not be expected to alter the function of LP85 protein and would, therefore, be expected to be functionally related.

“Host cell” refers to any eukaryotic or prokaryotic cell that is suitable for propagating and/or expressing a cloned gene contained on a vector that is introduced into said host cell by, for example, transformation or transfection, or the like.

The term “LP85” may refer to a nucleic acid, gene, cDNA (e.g. SEQ ID NO:1, 3, or 5), as well as to any polypeptide sequence (e.g., SEQ ID NO:2, 4, 6, or any fragments, analogs, or derivatives thereof). The term “LP85 protein” or “LP85 polypeptide” without further limitation encompasses native LP85 as shown in SEQ ID NO:2 and fragments thereof including, but not limited to, the mature form of LP85 polypeptide (predicted to be amino acids 19 through 370 of SEQ ID NO:2; also, referred to herein as LP85-N-18).

“LP85N shortest” specifically refers to a N-terminal fragment of LP85 comprising residues from about 1 through about 140 as shown in SEQ ID NOS:2, 4,and 6. LP85N shortest does not comprise the PDGF-like domain of LP85 and therefore does not exhibit the PDGF domain like activity, (e.g. the ability to modulate Map Kinase activity, stimulate BalbC cell growth, induce mitogenic activity in endothelial cells, skeletal muscle cells, smooth muscle cells, fibroblast cells, osteoblasts, and/or stimulate bone growth) exhibited by LP85 and other active fragments thereof.

“LP85N shorter” specifically refers to a N-terminal fragment of LP85 comprising residues from about 1 through about 175 of SEQ ID NO:2. LP85N shorter does not comprise the PDGF-like domain of LP85 and, therefore, does not exhibit the PDGF domain like activity (e.g. the ability to modulate Map Kinase activity, stimulate BalbC cell growth, induce mitogenic activity in endothelial cells, skeletal and smooth muscle cells, fibroblast cells, osteoblasts, and/or otherwise stimulate bone growth) exhibited by LP85 and other active fragments thereof.

“LP85N short” specifically refers to a N-terminal fragment of LP85 comprising residues from about 1 through about 249 of SEQ ID NO: 2. LP85N short does not comprise. the PDGF-like domain of LP85 and therefore does not exhibit the PDGF domain like activity, (e.g. the ability to modulate Map Kinase activity, stimulate BalbC cell growth, induce mitogenic activity in endothelial cells, skeletal cells, smooth muscle cells, fibroblast cells, osteoblasts, and/or otherwise stimulate bone growth) exhibited by LP85 and other active fragments thereof.

“LP8C short” specifically refers to a C-terminal fragment of LP85 comprising residues from about 250 through about 370 of SEQ ID NO: 2. LP85C short comprise the PDGF-like domain of LP85 and therefore exhibit the PDGF domain like activity, (e.g. the ability to modulate Map Kinase activity, stimulate BalbC cell growth, induce mitogenic activity in endothelial cells, skeletal cells, smooth muscle cells, fibroblast cells, osteoblasts, and/or otherwise stimulate bone growth) exhibited by LP85 and other active fragments thereof.

As used herein “half-life” refers to the time required for approximately half of the molecules making up a population of said molecules to be cleaved in vitro or in vivo. More specifically, “plasma half-life” refers to the time required for approximately half of the molecules making up a population of said molecules to be removed from circulation or be, otherwise, rendered inactive in vivo.

The term “homolog” or “homologous” designates a relationship of partial identity or similarity of sequence between nucleic acid molecules or protein molecules at one or more regions within said molecules. As used herein, the term “sufficiently homologous” refers to a first amino acid or nucleotide sequence which contains a sufficient or minimum number of identical or or related amino acid substitutions (for related amino acids see Table 1 for conservative substitutions and discussion of groups, infra.) or nucleotides to a second amino acid or nucleotide sequence such that the first and second amino acid or nucleotide sequences have a common structural domain and/or common functionality. Preferably, a sufficiently homologous polypeptide comprises a defined region having at least about 85% homology, more preferably at least about 90% homology, more preferably at least about 95% homology, more preferably at least about 96% homology, more preferably at least about 97% homology, more preferably at least about 98% homology, more preferably at least about 99% homology, and most preferably 100% homology to the entire region as defined. Preferably, a sufficiently homologous polynucleotide comprises a polynucleotide extending over a defined length having at least about 85% homology, more preferably at least about 90% homology, more preferably at least about 95% homology, more preferably at least about 96% homology, more preferably at least about 97% homology, more preferably at least about 98% homology, more preferably at least about 99% homology, and most preferably 100% amino acid homology over the entire region as defined.

The term “hybridization” as used herein refers to a process in which a single-stranded nucleic acid molecule joins with a complementary strand through nucleotide base pairing. “Selective hybridization” refers to hybridization under conditions of high stringency. The degree of hybridization depends upon, for example, the degree of homology or relatedness, the stringency of hybridization, and the length of hybridizing strands.

The term “inhibit” or “inhibiting” includes the generally accepted meaning, which includes prohibiting, preventing, restraining, slowing, stopping, or reversing progression or severity of a disease or condition.

In the present disclosure, “isolated” refers to material removed from its original environment (e.g., the natural environment if it is naturally occurring), and thus is altered “by the hand of man” from its natural state. For example, the term “isolated” in reference to a polypeptide refers to a polypeptide that has been identified and separated and/or recovered from at least one contaminant from which it has been produced. Contaminants may include cellular components, such as enzymes, hormones, and other proteinaceous or non-proteinaceous solutes. Ordinarily, however, isolated polypeptides will be prepared by at least one purification step.

The term “isolated” in reference to a nucleic acid compound refers to any specific RNA or DNA molecule, however constructed or synthesized or isolated, which is locationally distinct from its natural location. For example, an isolated polynucleotide could be part of a vector or a composition of matter, or could be contained within a cell, and still be “isolated” because that vector, composition of matter, or particular cell is not the original environment of the polynucleotide.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. Ordinarily, an isolated antibody is prepared by at least one purification step. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or preferably, silver stain.

An “isolated antibody” is also intended to mean an antibody that is substantially purified from other antibodies having different antigenic specificities. An isolated antibody that specifically binds LP85 epitopes may bind LP85 homologous molecules from other species.

The term “isolated” may be used interchangeably with the phrases “substantially pure” or “substantially purified” in reference to a macromolecule that is separated from other cellular and non-cellular molecules, including other proteins, lipids, carbohydrates or other materials with which it is naturally associated when produced recombinantly or synthesized without any general purifying steps. A “substantially pure” or “isolated” protein as described herein could be prepared by a variety of techniques well known to the skilled artisan. In preferred embodiments, a polypeptide will be isolated or substantialy purified upon purification (1) to greater than 85% by weight of polypeptide to the weight of total protein as determined by the Lowry method, and most preferably to more than 95% by weight of polypeptide to the weight of total protein, (2) to a degree sufficient to obtain at least 15 residues of N-terminal or internal amino acid sequence by use of a spinning cup sequenator, or (3) to apparent homogeneity by SDS-PAGE under reducing or nonreducing conditions using Coomassie Blue, or preferably, silver stain, such that the major band constitutes at least 85%, and, more preferably 95%, of stained protein observed on the gel.

A “nucleic acid probe” or “probe” as used herein is a labeled nucleic acid compound which hybridizes with another nucleic acid compound. “Nucleic acid probe” means a single stranded nucleic acid sequence that will combine with a complementary or partially complementary single stranded target nucleic acid sequence to form a double-stranded molecule. A nucleic acid probe may be an oligonucleotide or a nucleotide polymer. A probe will usually contain a detectable moiety which may be attached to the end(s) of the probe or be internal to the sequence of the probe.

The terms “ortholog”, “orthologue”, or “orthologous” refers to two or more genes or proteins from different organisms that exhibit sequence homology.

The terms “paralog”, “paralogue” or “paralogous” refers to two or more genes or proteins within a single organism that exhibit sequence homology.

The term “plasmid” refers to an extrachromosomal genetic element. The plasmids disclosed herein are commercially available, publicly available on an unrestricted basis, or can be constructed from readily available plasmids in accordance with published procedures.

A “primer” is a nucleic acid fragment which functions as an initiating substrate for enzymatic or synthetic elongation of, for example, a nucleic acid molecule.

The term “resistant” or more specifically “protease-resistant” or “glycosylation resistant” refers to a LP85 analog that is more resistant to proteolysis or glycosylation relative to native LP85 as shown in SEQ ID NO:2. Protease or glycosylation resistant analogs may differ from LP85 by one or more amino acid substitutions, deletions, inversions, additions, and/or other changes at any site susceptible to proteolysis or glycosylation. The term “resistant” contemplates degrees of resistance to at each of the different susceptible sites from complete resistance to partial resistance. Thus, a “substantially resistant” analog shows a degree of resistance a particular susceptible position such that the number of analogs cleaved or glycosylated at any particular position is at least about 25% fewer than the number of native LP85 molecules cleaved or glycosylated when similarly treated. Preferably a substantially protease resistant LP85 analog possesses a half-life that is at least about 2-fold greater than the corresponding native LP85 polypeptide. Similarly, a glycosylation resistant LP85 analog exhibits a clearance rate that is at least about 2-fold slower than the clearance rate of the corresponding native LP85 polypeptide

Susceptibility to proteolysis will depend on such factors as the amino acid sequence at or near the recognition site of the particular proteolytic enzyme involved, and on the physical and chemical environment in which a sample protein is located. Factors such as these can affect the KM and/or rate of proteolysis by a proteolytic enzyme. The charge density and steric properties operative at the enzymes active site will also determine the degree to which proteolysis occurs.

Susceptibility to glycosylation will depend on the amino acid sequence and the presence or absence of

The term “promoter” refers to a nucleic acid sequence that directs transcription, for example, of DNA to RNA. An inducible promoter is one that is regulatable by environmental signals, such as carbon source, heat, or metal ions, for example. A constitutive promoter generally operates at a constant level and is not regulatable.

“Recombinant DNA cloning vector” as used herein refers to any autonomously replicating agent, including, but not limited to, plasmids and phages, comprising a DNA molecule to which one or more additional DNA segments can or have been incorporated.

The term “recombinant DNA expression vector” or “expression vector” as used herein refers to any recombinant DNA cloning vector, for example a plasmid or phage, in which a promoter and other regulatory elements are present thereby enabling transcription of an inserted DNA, which may encode a protein.

The term “stability” in reference to a LP85 polypeptide and/or LP85 analog may refer to its half-life in vivo, in serum, and/or in solution.

The term “stringency” refers to hybridization conditions. High stringency conditions disfavor non-homologous base pairing. Low stringency conditions have the opposite effect. Stringency may be altered, for example, by temperature and salt concentration.

“Low stringency” conditions comprise, for example, a temperature of about 37° C. or less, a formamide concentration of less than about 50%, and a moderate to low salt (SSC) concentration; or, alternatively, a temperature of about 50° C. or less, and a moderate to high salt (SSPE) concentration, for example 1M NaCl.

“High stringency” conditions comprise, for example, a temperature of about 42° C. or less, a formamide concentration of less than about 20%, and a low salt (SSC) concentration; or, alternatively, a temperature of about 65° C., or less, and a low salt (SSPE) concentration. For example, high stringency conditions comprise hybridization in 0.5M NaHPO_{4, 7}% sodium dodecyl sulfate (SDS), 1 mM EDTA at 65° C. (Ausubel, F. M. et al. Current Protocols in Molecular Biology, Vol. I, 1989; Green Inc. New York, at 2.10.3).

“SSC” comprises a hybridization and wash solution. A stock 20×SSC solution contains 3M sodium chloride, 0.3M sodium citrate, pH 7.0.

“SSPE” comprises a hybridization and wash solution. A 1×SSPE solution contains 180 mM NaCl, 9 mM Na₂HPO₄, 0.9 μM NaH₂PO₄ and 1 mM EDTA, pH 7.4.

Pharmaceutical Terms

The term “administer” or “administering” means to introduce by any means a therapeutic agent into the body of a mammal in order to prevent or treat a disease or condition.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Administration “in combination with” one or more further therapeutic agents includes simultaneous (concurrent) and consecutive administration in any order.

A “biologically-effective amount” is the minimal amount of a compound or agent that is necessary to impart a biological consequence to the extent that the biological consequence is measurable either directly or indirectly. Such determinations are routine and within the skill of an ordinarily skilled artisan.

A “therapeutically-effective amount” is the minimal amount of a compound or agent that is necessary to impart therapeutic benefit to a mammal. By administering graduated levels of a LP85 polypeptide or LP85 analog to a mammal in need thereof, a clinician skilled in the art can determine the therapeutically effective amount of the LP85 polypeptide or LP85 analog required for administration in order to treat or prevent the diseases, condition, disorders, and/or at least one symptom thereof, discussed herein. Such determinations are routine in the art and within the skill of an ordinarily skilled clinician.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS™.

“Pharmaceutically acceptable salt” includes, but is not limited to, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “mammal” as used herein refers to any mammal, including humans, domestic and farm animals, and zoo, sports or pet animals, such as cattle (e.g. cows), horses, dogs, sheep, pigs, rabbits, goats, cats, and non-domesticated animals like mice and rats. In a preferred embodiment of the present invention, the mammal being treated or administered to is a human or mouse.

A “small molecule” is defined herein to have a molecular weight below about 500 daltons.

The terms “treating”, “treatment” and “therapy” as used herein refer to curative therapy, prophylactic therapy, and preventative therapy. An example of “preventative therapy” is the prevention or lessening of a targeted disease or related condition thereto. Those in need of treatment include those already with the disease or condition as well as those prone to have the disease or condition to be prevented. The terms “treating”, “treatment”, and “therapy” as used herein also describe the management and care of a mammal for the purpose of combating a disease, or related condition, and includes the administration of LP85 polypeptides or LP85 analogs to alleviate the symptoms or complications of said disease, condition. Treating as used herein also includes the administration of the protein for cosmetic purposes.

All references herein to a disease, condition, or disorder are contemplated to encompass the other diseases, conditions, disorders, and/or symptoms generally associated with that particular disease, condition, or disorder by the medical community.

A “therapeutically-effective amount” is the minimal amount of a compound or agent that is necessary to impart therapeutic benefit to a mammal. By administering graduated levels of a LP85 polypeptide or LP85 analog to a mammal in need thereof, a clinician skilled in the art can determine the therapeutically effective amount of the LP85 polypeptide or LP85 analog in order to treat or prevent a particular disease condition, or disorder when it is administered, such as intravenously, subcutaneously, intraperitoneally, orally, or through inhalation. The precise amount of the compound required to be therapeutically effective will depend upon numerous factors, e.g., such as the specific binding activity of the compound, the delivery device employed, physical characteristics of the compound, purpose for the administration, in addition to patient specific considerations. The amount of a compound that must be administered to be therapeutically effective are routine in the art and within the skill of an ordinarily skilled clinician.

The term “vector” as used herein refers to a nucleic acid compound used for introducing exogenous or endogenous DNA into host cells. A vector comprises a nucleotide sequence which may encode one or more protein molecules. Plasmids, cosmids, viruses, and bacteriophages, in the natural state or which have undergone recombinant engineering, are examples of commonly used vectors.

The various restriction enzymes disclosed and described herein are commercially available and the manner of use of said enzymes including reaction conditions, cofactors, and other requirements for activity are well known to one of ordinary skill in the art. Reaction conditions for particular enzymes were carried out according to the manufacturer's recommendation.

Applicants have shown that mRNA transcripts encoding LP85 polypeptides are expressed in multiple tissues including, but not limited to, epithelial cells including, but not limited to skin, cervix, vagina, and tonsils, and smooth muscle cells, including, but not limited to those found, in heart tissue. Importantly, LP85 encoding MRNA transcripts are also highly expressed in osteoblasts from fetal baboon. LP85 polypeptides were also shown to stimulate the proliferation, mitogenesis, and/or growth of multiple cell types, including, but not limited to, bone, heart, and epithelial cells. LP85 also appears to be susceptible to cleavage between the lysine residue at position 31 and the alanine residue at position 32 SEQ ID NO:2. Cleavage products resulting from cleavage at this site comprise residues 1-31 and 32-370 as shown in SEQ ID NO:2. The cleavage products comprising amino acid residues 32-370 comprise the complete N-terminal CUB domain (which is thought to target the molecule appropriately in vivo) and the complete C-terminal PDGF-like domain. Therefore, cleavage products resulting from cleavage at this site will likely comprise amino acid residues 32-370, termed hereinafter as the “LP85 metabolite”. This LP85 metabolite retains substantially the same biological activity as native LP85 molecules that are not cleaved between positions 31 and 32. Thus, one embodiment of the present invention relates to an LP85 metabolite comprising amino acid residues 32-370 and its use in treating and/or preventing the disorders described herein as well as other related disorders.

Applicants have also discovered that LP85 polypeptides are susceptible to cleavage between the arginine residue at position 249 and the serine residue at position 250 as shown in SEQ ID NO:2. More specifically, when LP85 is expressed in CHO-DG44 or CHO-K1 cells, the protein is almost completely cleaved at or near this site after six days. Cleavage products obtained from this reaction consist of LP85 polypeptides comprising residues around 1 through 249 and around 250 through around 370 as shown in SEQ ID NO:2. Cleavage products comprising amino acid residues around 1 through around 249 or alternatively around 32 through around 249 (if also cleaved at Lys31 site) comprise the complete N-terminal CUB domain (which is thought to be necessary to target the molecule appropriately in vivo) while the C-terminal cleavage product comprising residues around 250 through around 370 as shown in SEQ ID NO:2 includes the complete C-terminal PDGF-like domain. Therefore, the C-terminal fragment generated from cleavage at the Arg249 will likely result in non-specific PDGF domain-like biological activity as compared to LP85 analogs which are less susceptible to cleavage at Arg249 and, therefore, will more likely contain both the CUB domain and PDGF-like domain. When Arg249 was replaced with a glutamine residue, the LP85 analog expressed by cells transiently transformed with an expression vector encoding the LP85 analog remained substantially intact as determined by Western Blot analysis of the six day culture media.

The LP85 metabolite as disclosed herein as well as other LP85 fragments comprising amino acid residue around 250 through around 370 may be produced in vitro by treating a LP85 polypeptide with a trypsin-like protease. Furthermore, Applicants have observed that the LP85 metabolite can be produced upon limited Lys-C enzyme digestions of LP85 polypeptides including this cleavage site. Alternatively, the metabolite and the C-terminal fragments can be produced through recombinant DNA mutagenesis approaches known in the art or as described in the later section. Finally, the metabolite and the C-terminal fragments can be produced through synthetic peptide synthesis approaches known in the art.

One embodiment of the present invention relates to methods of making and using LP85 and LP85 analogs which retain the biological activity of the native LP85 but are more resistant to proteolysis around or between residues 31 and 32 of SEQ ID NO:2 and/or around or between residues 249 and 250 of SEQ ID NO:2. Biological activity relates to the capacity of a particular LP85 polypeptide or LP85 analog thereof to induce biological consequences similar to those discussed herein, including in vivo and/or in vitro induction of mitogenic activity in endothelial cells, skeletal and smooth muscle cells, fibroblast cells, osteoblasts, and/or bone growth as well as inhibition of IL-1β-induced inhibition of proteoglycan synthesis.

LP85 analogs of the present invention comprise one or more changes, such as amino acid substitutions, deletions, inversions, additions, or changes in glycosylation sites or patterns and/or combinations thereof that prevent or diminish proteolysis, and/or the rate thereof, around or between residues 31 and 32 of SEQ ID NO:2 and/or around or between residues 249 and 250 of SEQ ID NO:2. Preferably these changes occur at or near the protease recognition sequence of LP85; most preferably, at or near the dipeptide sequence at positions 30 and 31 of SEQ ID NO:2 and/or at or near the dipeptide sequence at positions 248 and 249 of SEQ ID NO:2. As the skilled artisan understands, residues at or near a recognition site can also affect the susceptibility of the substrate protein to proteolysis by altering the charge milieu at the active site and/or by creating alterations by steric hindrance in the region of the active site.

Therefore, the invention contemplates LP85 analogs comprising amino acid changes in LP85 or fragments thereof. Preferably the amino acid changes in LP85 analogs as compared to LP85 polypeptides occur in the regions from about position 26 through position 35 of SEQ ID NO:2, from about position 246 through position 255 of SEQ ID NO:2, or the corresponding regions of SEQ ID NO:4 or 6. More preferred protease resistant analogs of LP85 include LP85 analogs which comprise amino acid substitutions, deletions, inversions, additions, and/or changes in glycosylation sites, or patterns, the region from about position 26 through about position 34, and/or the region from about position 245 through about position 270 (all positions are in reference to SEQ ID NO:2). Most preferably, LP85 protease-resistant analogs comprise amino acid between positions 31 and 32 of SEQ ID NO:2 and/or between positions 249 and 250 of SEQ ID NO:2. Also contemplated by the present invention are protease-resistant LP85 analogs comprising substitutions, deletions, insertions, inversions, additions, or changes in glycosylation sites or patterns that occur outside the preferred windows described above.

It is also preferred that a protease resistant LP85 fragment or LP85 analog of the present invention display a half-life at least 10%, 20%, 30%, 40%, 50%, 60%, 70%, 80%, 90%, or 100% greater than native LP85 or the corresponding fragment thereof, as determined by the relative quantity of intact molecules to smaller digestion products (e.g. fragments 1-31 and 32-370 of SEQ ID NO:2). More preferably, a protease-resistant LP85 fragment or LP85 analog of the present invention displays a half-life between 100% and 200%, 300%, 400%, or 500% greater than native LP85 or the corresponding fragment thereof. Most preferably, a protease resistant LP85 analog possesses a half-life that is from about 10-fold greater to about 100-fold or greater than the LP85 or corresponding fragment thereof. Any method known in the art or any other suitable method for making such a qualitative and/or quantitative assessment of said relative quantities can be used (e.g., polyacrylamide gel electrophoresis).

In one embodiment of the present invention, a single amino acid change is made within at least one of these proteolytically susceptible regions; alternatively, at least two changes are made within at least one of these regions; alternatively, at least three changes are made within at least one of these regions; alternatively, at least four changes are made within at least one of these regions. As the skilled artisan understands, many substitutions, and/or other changes to a protein's sequence or structure, can be made without substantially affecting the biological activity or characteristics of the polypeptide. For example, making conservative amino acid substitutions, or changing one amino acid for another from the same class of amino acids, for example negatively charged residues, positively charged residues, polar uncharged residues, and non-polar residues, or any other classification acceptable in the art are often without effect on function. LP85 analogs comprising additional modifications made entirely in accordance with art recognized substitutability of amino acids and/or entirely to preserve the destruction of reactive site identified and disclosed herein (See e.g. M. Dayhoff, In Atlas of Protein Sequence and Structure, Vol. 5, Supp. 3, pgs 345-352, 1978; known protease motifs, etc.) are also contemplated as being encompassed by the present invention if the additionally modified LP85 analog retains substantially similar biological activities and pharmaceutically desirable properties of the LP85 analogs disclosed herein.

One factor that can be considered in making such changes is the hydropathic index of amino acids. The importance of the hydropathic amino acid index in conferring interactive biological function on a protein has been discussed by Kyte and Doolittle (1982, J. Mol. Biol., 157: 105-132). It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide, polypeptide, or protein can be substituted for other amino acids having a similar hydropathic index or score and produce a resultant peptide having similar biological activity, i.e., which still retains biological functionality. In making such changes, it is preferable that amino acids having hydropathic indices within +2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant peptide having similar biological activity, i.e., still retaining correct biological function. In making such changes, amino acids having hydropathic indices within ±2 are preferably substituted for one another, those within ±1 are more preferred, and those within ±0.5 are most preferred.

As outlined above, amino acid substitutions can be incorporated into the LP85 analogs of the present invention based on the relative similarity of the amino acid side-chain substituents, for example, their hydrophobicity, hydrophilicity, charge, size, etc., to yield a substantially similar analog having substantially similar properties. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine. To the extent that such modifications are made to the analogs of the present invention and the modified analogs retain substantially the same activity and substantially the same pharmaceutically desirable properties of the LP85 disclosed herein they are contemplated as being within the scope of the present invention. The utility of such additionally modified LP85 analogs can be determined without undue experimentation by, for example, the methods described herein.

In another embodiment, the invention relates to a LP85 analog comprising one or more amino acid substitution(s) in the region 26-35 of SEQ ID NO:2, and/or amino acids 245-257 of SEQ ID NO:2.

In another embodiment, the invention relates to a LP85 analog comprising an amino acid substitution(s) in the region comprising amino acids 26-35 of SEQ ID NO:2, selected from the group consisting of:

a. Gin at position 26 as shown in SEQ ID NO:2 is replaced by any naturally occurring amino acid other than Gln, Lys, and Arg;

b. Ser at position 27 as shown in SEQ ID NO:2 is replaced by any naturally occurring amino acid other than Ser, Lys, and Arg;

c. Ala at position 28 as shown in SEQ ID NO:2 is replaced by any naturally occurring amino acid other than Ala, Lys, and Arg;

d. Ser at position 29 as shown in SEQ ID NO:2 is replaced by any naturally occurring amino acid other than Ser, Lys, and Arg;

e. Ile at position 30 as shown in SEQ ID NO:2 is replaced by any naturally occurring amino acid other than Ile, Lys, and Arg;

f. Lys at position 31 as shown in SEQ ID NO:2 is replaced by any naturally occurring amino acid other than Lys and Arg;

g. Ala at position 32 as shown in SEQ ID NO:2 is replaced by any naturally occurring amino acid other than Ala, Lys, and Arg;

h. Leu at position 33 as shown in SEQ ID NO:2 is replaced by any naturally occurring amino acid other than Leu, Lys, and Arg;

i. Arg at position 34 as shown in SEQ ID NO:2 is replaced by any naturally occurring amino acid other than Lys;

j. Asn at position 35 as shown in SEQ ID NO:2 is replaced by any naturally occurring amino acid other than Asn, Lys, and Arg.

A number of other positively charged amino acids such as Arg and Lys are found in the vicinity of Arg249 as shown in SEQ ID No:2. This apparent hinge region is likely to be flexible, making other Arg and Lys residues in the region susceptible to proteolysis too. Therefore, another embodiment of the present invention includes a LP85 analog comprising an amino acid substitution in the region comprising amino acids 245-257 of SEQ ID NO:2 wherein said substitution is selected from the group consisting of:

a. Arg at position 245 as shown in SEQ ID NO:2 is replaced by any amino acid other than Lys;

b. Arg at position 249 as shown in SEQ ID NO:2 is replaced by neutral or negatively charged amino acids such as Glu, Gln, or Ala;

c. Arg at position 254 as shown in SEQ ID NO:2 is replaced by any other amino acids except for Lys;

d. Lys at position 255 as shown in SEQ ID NO:2 is replaced by any other amino acidsn except for Arg; and

e. Lys at position 257 as shown in SEQ ID NO:2 is replaced by any other amino acids except for Arg.

The term “N-glycosylated polypeptide” refers to polypeptides having one or more NXS/T motifs in which the nitrogen atom in the side chain amide of the asparagine is covalently bonded to a glycosyl group. “X” refers to any naturally occurring amino acid residue except proline. The “naturally occurring amino acids” are glycine, alanine, valine, leucine, isoleucine, proline, serine, threonine, cysteine, methionine, lysine, arganine, glutamic acid, asparatic acid, glutamine, asparagine, phenylalanine, histidine, tyrosine and tryptophan. N-glycosylated proteins are optionally O-glycosylation.

The term “O-glycosylated polypeptide” refers to polypeptides having one or more serines and/or threonine in which the oxygen atom in the side chain is covalently bonded to a glycosyl group. O-Glycosylated proteins are optionally N-glycosylation. Glycosylated polypeptides can be prepared recombinantly by expressing a gene encoding a polypeptide in a suitable mammalian host cell, resulting in glycosylation of side chain amides found in accessible NXT/S motifs on the polypeptide surface and/or of side chain alcohols of surface accessible serines and threonines. Specific procedures for recombinantly expressing genes in mammalian cells are provided hereinbelow. Other procedures for preparing glycosylated proteins are disclosed in EP 640,619 to Elliot and Burn, the entire teachings of which are incorporated herein by reference. Unglycosylated polypeptides can be prepared recombinantly by expressing a gene encoding a polypeptide in a suitable prokaryotic host cell.

Another example of a LP85 analog encompassed by the present invention is a LP85 analog of the present further comprising at least one oligopeptide or amino acid added onto the N-terminus and/or C-terminus. An “oligopeptide” is a chain of from two to about twenty-five amino acids connected at their N- and C-termini by peptide bonds. Suitable oligopeptides and amino acids are those which do not significantly decrease the activity of LP85 or corresponding fragment thereof, do not substantially detract from the pharmaceutical and pharmacological properties of LP85 and do not significantly decrease the in vivo half-live of LP85. Preferred examples include leader sequences found in native LP85, such as MHRLIFVYTL ICANFCSC(SEQ ID NO.:2).

The LP85 analogs of the present invention also include modified and unmodified LP85 analogs of the present invention further comprising one or more polyethylene glycol groups (hereinafter “PEG” groups). The PEG groups can be bonded to the N-terminus or to amine groups or thiol groups in the amino acid side chain(s) of LP85 analogs. Suitable PEG groups are known in the art. Suitable PEG groups generally have a molecular weight between about 5000 and 40,000 atomic mass units. Procedures for preparing PEGylated polypeptides are disclosed in Mumtaz and Bachhawat, Indian Journal of Biochemistry and Biophysics 28:346 (1991) and Francis et al., International Journal of Hematology 68:1 (1998), the entire teachings of which are incorporated herein by reference.

The LP85 analogs of the present invention can also be expressed in a modified form, such as a fusion protein or a “tagged” protein. LP85 analog fusion proteins represent a hybrid protein molecule comprising a translational fusion or enzymatic fusion in which at least LP85 fragment or LP85 analog of the present invention are covalently linked on a single polypeptide chain. Human serum albumin, the C-terminal domain of thrombopoietin, the C-terminal extension peptide of hCG, and/or a Fc fragment are examples of proteins which can be fused with LP85 analogs or LP85 fragments of the present invention. As used herein, “Fc fragment” of an antibody has the meaning commonly given to the term in the field of immunology. Specifically, this term refers to an antibody fragment which binds complement and is obtained by removing the two antigen binding regions (the Fab Fragments) from the antibody. Thus, the Fc fragment is formed from approximately equal sized fragments from both heavy chains, which associate through non-covalent interactions and disulfide bonds. The Fc Fragment includes the hinge regions and extends through the C_{H}2 and C_{H}3 domains to the C-terminus of the antibody. Procedures for preparing fusion proteins are disclosed in EP394,827, Tranecker et al., Nature 331:84 (1988) and Fares, et al., Proc. Natl. Acad. Sci. USA 89:4304 (1992), the entire teachings of which are incorporated herein by reference.

Many fusion proteins can be secreted by virtue of heterologous secretion signals in regions that can be removed prior to final preparation of the polypeptide. Such methodologies are described in many standard laboratory manuals, such as Sambrook, supra, Chapters 17.29-17.42 and 18.1-18.74; Ausubel, supra, Chapters 16, 17 and 18, the entire relevant teachings of which are incorporated herein by reference.

In a preferred process for protein expression and subsequent purification, the LP85 gene can be modified at the 5′ end to incorporate several histidine residues at the amino terminus of the LP85 protein resulting from its expression. This “histidine tag” enables a single-step protein purification method referred to as “immobilized metal ion affinity chromatography” (IMAC), essentially as described in U.S. Pat. No. 4,569,794, which hereby is incorporated by reference. The IMAC method enables rapid isolation of substantially pure recombinant LP85 protein starting from a crude extract of cells that express a modified recombinant protein, as described above.

The LP85 analogs of the present invention can also be glycosylated or unglycosylated. A glycosylated polypeptide is modified with one or more monosaccharides or oligosaccharides. A monosaccharide is a chiral polyhydroxyalkanol or polyhydroxyalkanone which typically exists in hemiacetal form. An “oligosaccharide” is a polymer of from about 2 to about 18 monosaccharides which are generally linked by acetal bonds. One type of glycosyl group commonly found in glycosylated proteins is N-acetylneuraminic acid. A glycosylated polypeptide can be N-glycosylated and/or O-glycosylated, preferably N-glycosylated.

LP85 analogs of the present invention can easily be tested for biological activity and/or sensitivity to proteolysis as described herein and as otherwise known in the art. Biological activity can be assessed using either in vitro models (e.g., see Examples) or in vivo models as described herein (e.g., see Examples 5 and 14) or otherwise known in the art. The functionality of LP85 analogs are quantifiable using assays including, but not limited to, mitogenic assays (including, but not limited to those using endothelial cells, skeletal muscle cells, smooth muscle cells, fibroblast cells, osteoblasts), in vitro bone marker or reporter assays, or in vitro or in vivo bone growth or bone deterioration assays, for example.

Applicants have observed that native LP85 is rapidly cleared from blood circulation. The lone N-linked glycosylation site Asn276 was found to contain high mannose type structures. Various studies from literature have shown that mannose receptors rapidly eliminate glycoproteins and microorganism bearing high mannose type carbohydrate chains from blood circulation. Examples include t-PA (Bieseen et al., Circulation, 95, 46, (1997)), circulating C-terminal propeptide of type I procollagen (Smedsrod B et al., 1990, Biochem J., 271, 345, (1990)), and glycosylated human salivary amylase (Niesen TE et al., J Leukoc. Biol. 36, 307, (1984)). The applicants contemplate the high mannose type carbohydrate structure may be at least partly responsible for the observed rapid clearance of LP85. The present invention is contemplated to include LP85 analogs which comprise at least one amino acid substitution at or near regions thought to be susceptible to glycosylation. Preferred embodiments of the present invention are LP85 analogs which comprise at least one amino acid substitution at or near regions thought to be susceptible to N-linked glycosylation. Most preferred embodiments of the present invention, therefore, include LP85 analogs comprising amino acid substitutions at Asn276 wherein said subsitution is selected from the group consisting of:

a. Asn at position 276 as shown in SEQ ID NO:2 is replaced by a Gln;

b. Asn at position 276 as shown in SEQ ID NO:2 is replaced by a Asp;

c. Asn at position 276 as shown in SEQ ID NO:2 is replaced by a Glu;

d. Asn at position 276 as shown in SEQ ID NO:2 is replaced by a Thr;

e. Asn at position 276 as shown in SEQ ID NO:2 is replaced by a Ala;

f. Asn at position 276 as shown in SEQ ID NO:2 is replaced by a Ser;

g. Asn at position 276 as shown in SEQ ID NO:2 is replaced by a Gln; and

h. Ser at position 278 as shown in SEQ ID NO:2 is replaced by any naturally occurring amino acid other than Ser or Thr.

In addition, Tyr at position 277 can be replaced with proline and Val at position 279 can be replaced with proline to prevent glycosylation at Asn276.

Fragments of the LP85 proteins, and analogs thereof, may be generated by any number of suitable techniques, including chemical synthesis of any portion of SEQ ID NO:2, proteolytic digestion of LP85 polypeptides, or most preferably, by recombinant DNA mutagenesis techniques well known to the skilled artisan. See. e.g. K. Struhl, “Reverse biochemistry: Methods and applications for synthesizing yeast proteins in vitro,” Meth. Enzymol. 194, 520-535. For example, in a preferred method, a nested set of deletion mutations are introduced into a nucleic acid sequence encoding LP85 (e.g. nucleotides 114 through 1223 of SEQ ID NO:1) such that varying amounts of the protein coding region are deleted, either from the amino terminal end or from the carboxyl end of the protein molecule. This method can also be used to create internal fragments of the intact protein in which both the carboxyl and amino terminal ends are removed. Several appropriate nucleases can be used to create such deletions, for example Bal31, or in the case of a single stranded nucleic acid molecule, mung bean nuclease. For simplicity, it is preferred that the LP85 gene be cloned into a single-stranded cloning vector, such as bacteriophage M13, or equivalent. If desired, the resulting gene deletion fragments can be subcloned into any suitable vector for propagation and expression of said fragments in any suitable host cell.

Functional fragments of the proteins disclosed herein may be produced as described above, preferably using cloning techniques to engineer smaller versions of the intact gene, lacking sequence from the 5′ end, the 3′ end, from both ends, or from an internal site. Fragments may be tested for biological activity using any suitable assay, for example, the ability of a protein fragment to stimulate the proliferation, mitogenesis, and/or growth of multiple cell types, including, but not limited to, bone, heart, and epithelial cells, in vivo or in vitro.

Those skilled in the art will recognize that the LP85 gene could be obtained by a plurality of recombinant DNA techniques including, for example, hybridization, polymerase chain reaction (PCR) amplification, or de novo DNA synthesis. (See e.g., T. Maniatis et al. Molecular Cloning: A Laboratory Manual, 2d Ed. Chap. 14 (1989)).

Methods for constructing cDNA libraries in a suitable vector such as a plasmid or phage for propagation in prokaryotic or eukaryotic cells are well known to those skilled in the art. [See e.g. Maniatis et al. Supra]. Suitable cloning vectors are well known and are widely available.

The LP85 gene, or any fragment thereof, can be isolated from a tissue in which said gene is expressed, for example, placenta. In one method, mRNA is isolated, and first strand cDNA synthesis is carried out. A second round of DNA synthesis can be carried out for the production of the second strand. If desired, the double-stranded cDNA can be cloned into any suitable vector, for example, a plasmid, thereby forming a cDNA library. oligonucleotide primers targeted to any suitable region of SEQ ID NO:1 can be used for PCR amplification of LP85. See e.g. PCR Protocols: A Guide to Method and Application, Ed. M. Innis et al., Academic Press (1990). The PCR amplification comprises template DNA, suitable enzymes, primers, and buffers, and is conveniently carried out in a DNA Thermal Cycler (Perkin Elmer Cetus, Norwalk, Conn.). A positive result is determined by detecting an appropriately-sized DNA fragment following agarose gel electrophoresis.

Skilled artisans will recognize that the proteins of the present invention can be synthesized by a number of different methods, such as chemical methods well known in the art, including solid phase peptide synthesis or recombinant methods. Both methods are described in U.S. Pat. No. 4,617,149, incorporated herein by reference.

The principles of solid phase chemical synthesis of polypeptides are well known in the art and may be found in general texts in the area. See, e.g., H. Dugas and C. Penney, Bioorganic Chemistry (1981) Springer-Verlag, New York, 54-92. For example, peptides may be synthesized by solid-phase methodology utilizing an Applied Biosystems 430A peptide synthesizer (Applied Biosystems, Foster City, Calif.) and synthesis cycles supplied by Applied Biosystems. Additionally, peptides may be chemically ligated together by one skilled in the art of synthetic peptide synthesis.

The proteins of the present invention can also be produced by recombinant DNA methods using the cloned LP85 gene. Recombinant methods are preferred if a high yield is desired. Expression of the cloned gene can be carried out in a variety of suitable host cells, well known to those skilled in the art. For this purpose, the LP85 gene is introduced into a host cell by any suitable means, well known to those skilled in the art. While chromosomal integration of the cloned gene is within the scope of the present invention, it is preferred that the gene be cloned into a suitable extra-chromosomally maintained expression vector so that the coding region of the LP85 gene is operably-linked to a constitutive or inducible promoter.

The basic steps in the recombinant production of LP85 protein or fragment or analog thereof are:

a) constructing a natural, synthetic or semi-synthetic DNA encoding said LP85 protein or fragment or analog thereof;

b) integrating said DNA into an expression vector in a manner suitable for expressing the LP85 protein or fragment or analog thereof, either alone or as a fusion protein;

c) transforming or otherwise introducing said vector into an appropriate eukaryotic or prokaryotic host cell forming a recombinant host cell,

d) culturing said recombinant host cell in a manner to express the LP85 protein or fragment or analog thereof; and

e) recovering and substantially purifying the LP85 protein or fragment or analog thereof by any suitable means, well known to those skilled in the art.

Prokaryotes may be employed in the production of recombinant LP85 proteins or fragments or analogs thereof. For example, the Escherichia coli K12 strain 294 (ATCC No. 31446) is particularly useful for the prokaryotic expression of foreign proteins. Other strains of E. coli, bacilli such as Bacillus subtilis, enterobacteriaceae such as Salmonella typhimurium or Serratia marcescans, various Pseudomonas species and other bacteria, such as Streptomyces, may also be employed as host cells in the cloning and expression of the recombinant proteins of this invention.

Promoter sequences suitable for driving the expression of genes in prokaryotes include β-lactamase [e.g. vector GX2907, ATCC 39344, contains a replicon and β-lactamase gene], lactose systems [Chang et al., Nature (London), 275:615 (1978); Goeddel et al., Nature (London), 281:544 (1979)], alkaline phosphatase, and the tryptophan (trp) promoter system [vector pATH1 (ATCC 37695)], which is designed to facilitate expression of an open reading frame as a trpE fusion protein under the control of the trp promoter. Hybrid promoters such as the tac promoter (isolatable from plasmid pDR540, ATCC-37282) are also suitable. Still other bacterial promoters, whose nucleotide sequences are generally known, may be ligated to DNA encoding the protein of the instant invention, using linkers or adapters to supply any required restriction sites. Promoters for use in bacterial systems also will contain a Shine-Dalgarno sequence operably-linked to the DNA encoding the desired polypeptides. These examples are illustrative rather than limiting.

Vectors

Another aspect of the present invention relates to recombinant DNA cloning vectors and expression vectors comprising the nucleic acids encoding at least one LP85 fragments or LP85 analog of the present invention. The preferred nucleic acid vectors are those which comprise DNA sequences that encode residues from about 249 through about 370 of SEQ ID NO:2. More preferred recombinant DNA vectors comprise DNA sequences that encode residues from about 175 through about 370 of SEQ ID NO:2. Most preferred recombinant DNA vectors comprise DNA sequences that encode residues from about 19 through about 370 of SEQ ID NO:2.

The skilled artisan understands that choosing the most appropriate cloning vector or expression vector depends upon a number of factors including the availability of restriction enzyme sites, the type of host cell into which the vector is to be transfected or transformed, the purpose of the transfection or transformation (e.g., stable transformation as an extrachromosomal element, or integration into the host chromosome), the presence or absence of readily assayable or selectable markers (e.g., antibiotic resistance and metabolic markers of one type and another), and the number of copies of the gene desired in the host cell.

Vectors suitable to carry the nucleic acids of the present invention comprise RNA viruses, DNA viruses, lytic bacteriophages, lysogenic bacteriophages, stable bacteriophages, plasmids, viroids, and the like. The most preferred vectors are plasmids.

When preparing an expression vector the skilled artisan understands that there are many variables to be considered, for example, whether to use a constitutive or inducible promoter. The practitioner also understands that the amount of nucleic acid or protein to be produced dictates, in part, the selection of the expression system. Regarding promoter sequences, inducible promoters are preferred because they enable high level, regulatable expression of an operably-linked gene. The skilled artisan will recognize a number of suitable promoters that respond to a variety of inducers, for example, carbon source, metal ions, and heat. Other relevant considerations regarding an expression vector include whether to include sequences for directing the localization of a recombinant protein. For example, a sequence encoding a signal peptide preceding the coding region of a gene is useful for directing the extra-cellular export of a resulting polypeptide.

A suitable host cell for producing the LP85 fragments or LP85 analogs of the present invention is any eukaryotic cell that can accomodate high level expression of an exogenously introduced gene or protein, and that will secrete said protein. Transformed host cells may be cultured under conditions well known to skilled artisans such that a polypeptide as shown in either SEQ ID NO:2, 4, or 6 is expressed, thereby producing a recombinant LP85 protein in the recombinant host cell.

The proteins of this invention may be synthesized either by direct expression or as a fusion protein comprising the protein of interest as a translational fusion with another protein or peptide which may be removable by enzymatic or chemical cleavage. It is often observed in the production of certain peptides in recombinant systems that expression as a fusion protein prolongs the life span, increases the yield of the desired peptide, or provides a convenient means of purifying the protein. This is particularly relevant when expressing mammalian proteins in prokaryotic hosts. A variety of peptidases (e.g. enterokinase and thrombin) which cleave a polypeptide at specific sites or digest the peptides from the amino- or carboxy-termini (e.g. diaminopeptidase) of the peptide chain are known. Furthermore, particular chemicals (e.g. cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites (See e.g., P. Carter, “Site Specific Proteolysis of Fusion Proteins”, Chapter 13, in Protein Purification: From Molecular Mechanisms to Large Scale Processes, American Chemical Society, Washington, D.C. (1990)).

In addition to prokaryotes, a variety of amphibian expression systems such as frog oocytes, and mammalian cell systems can be used. The choice of a particular host cell depends to some extent on the particular expression vector used. Exemplary mammalian host cells suitable for use in the present invention include HepG-2 (ATCC HB 8065), CV-1 (ATCC CCL 70), LC-MK2 (ATCC CCL 7.1), 3T3 (ATCC CCL 92), CHO-K1 (ATCC CCL 61), HeLa (ATCC CCL 2), RPMI8226 (ATCC CCL 155), H4IIEC3 (ATCC CCL 1600), C127I (ATCC CCL 1616), HS-Sultan (ATCC CCL 1484), and BHK-21 (ATCC CCL 10), for example.

A wide variety of vectors are suitable for transforming mammalian host cells. For example, the pSV2-type vectors comprise segments of the simian virus 40 (SV40) genome required for transcription and polyadenylation. A large number of plasmid pSV2-type vectors have been constructed, such as pSV2-gpt, pSV2-neo, pSv2-dhfr, pSV2-hyg, and pSV2-b-globin, in which the SV40 promoter drives transcription of an inserted gene. These vectors are widely available from sources such as the American Type Culture Collection (ATCC), 12301 Parklawn Drive, Rockville, Md., 20852, or the Northern Regional Research Laboratory (NRRL), 1815 N. University Street, Peoria, Ill., 61604.

Promoters suitable for expression in mammalian cells include the SV40 late promoter, promoters from eukaryotic genes, such as, for example, the estrogen-inducible chicken ovalbumin gene, the interferon genes, the glucocorticoid-inducible tyrosine aminotransferase gene, the thymidine kinase gene promoter, and the promoters of the major early and late adenovirus genes and the cytomegalovirus promoter.

Plasmid pRSVcat (ATCC 37152) comprises portions of a long terminal repeat of the Rous Sarcoma virus, a virus known to infect chickens and other host cells. This long terminal repeat contains a promoter which is suitable for use in the vectors of this invention. H. Gorman et al., Proc. Nat. Acad. Sci. (USA), 79, 6777 (1982). The plasmid pMSVi (NRRL B-15929) comprises the long terminal repeats of the Murine Sarcoma virus, a virus known to infect mouse and other host cells. The mouse metallothionein promoter has also been well characterized for use in eukaryotic host cells and is suitable for use in the present invention. This promoter is present in the plasmid pdBPV-MMTneo (ATCC 37224) which can serve as the starting material for the construction of other plasmids of the present invention.

Transfection of mammalian cells with vectors can be performed by a plurality of well known processes including, but not limited to, protoplast fusion, calcium phosphate co-precipitation, electroporation and the like. See, e.g., Maniatis et al., supra.

Some viruses also make appropriate vectors. Examples include the adenoviruses, the adeno-associated viruses, the vaccinia virus, the herpes viruses, the baculoviruses, and the rous sarcoma virus, as described in U.S. Pat. No. 4,775,624, incorporated herein by reference.

Eukaryotic microorganisms such as yeast and other fungi are also suitable host cells. The yeast Saccharomyces cerevisiae is the preferred eukaryotic microorganism. Other yeasts such as Kluyveromyces lactis and Pichia pastoris are also suitable. For expression in Saccharomyces, the plasmid YRp7 (ATCC-40053), for example, may be used. See, e.g., L. Stinchcomb et al., Nature, 282, 39 (1979); J. Kingsman et al., Gene, 7, 141 (1979); S. Tschemper et al., Gene, 10, 157 (1980). Plasmid YRp7 contains the TRP1 gene which provides a selectable marker for use in a trp1 auxotrophic mutant.

An expression vector carrying a CDNA encoding a LP85 fragment or LP85 analog of the present invention is transformed or transfected into a suitable host cell using standard methods. Cells that contain the vector are propagated under conditions suitable for expression of the recombinant LP85 fragment or LP85 analog. For example, if the recombinant gene has been placed under the control of an inducible promoter, suitable growth conditions would incorporate the appropriate inducer. The recombinantly-produced protein may be purified from cellular extracts of transformed cells by any suitable means.

The cDNA molecules that encode LP85 functional fragments and/or LP85 analogs, may be produced by chemical synthetic methods or generated using a conventional DNA synthesizing apparatus, such as the Applied Biosystems Model 380A or 380B DNA synthesizers (Applied Biosystems, Inc., 850 Lincoln Center Drive, Foster City, Calif. 94404) using phosphoramidite chemistry, thereafter ligating the fragments so as to reconstitute the entire gene. Alternatively, phosphotriester chemistry may be employed to 1synthesize the nucleic acids of this invention. (See, e.g., M. J. Gait, ed., Oligonucleotide Synthesis, A Practical Approach, (1984)). In any event, the synthesis of nucleic acids is well known in the art. See, e.g., E. L. Brown, R. Belagaje, M. J. Ryan, and H. G. Khorana, Methods in Enzymology, 68:109-151 (1979).

In an alternative methodology, namely PCR, the DNA sequences encoding the LP85 analogs of the present invention can be produced, for example, starting with a cDNA preparation (e.g. cDNA library) derived from a tissue that expresses the LP85 gene, suitable oligonucleotide primers complementary to SEQ ID NO:1 or to any sub-region therein, are prepared as described in U.S. Pat. No. 4,889,818, hereby incorporated by reference. Other suitable protocols for the PCR are disclosed in PCR Protocols: A Guide to Method and Applications, Ed. Michael A. Innis et al., Academic Press, Inc. (1990). Using PCR, any region of the LP85 gene can be targeted for amplification such that appropriate sequences are amplified.

In another embodiment, the present invention provides methods for using LP85 analogs to treat or prevent diseases and/or conditions related to abnormal musculoskeletal structure, function, or metabolism including, but not limited to, osteoporosis, osteopenia, sarcopenia, various forms of arthritis, tissue atrophy, peridontal disease, wound healing, traumatized connective tissues, grafted connective tissues and/or transplanted organs wherein said method comprises the administration of a therapeutically effective amount of at least one LP85 analog.

In another embodiment, the present invention relates to a method for treating and/or preventing musculoskeletal conditions such as osteopenia, osteoarthritis, sarcopenia, or osteoporosis, comprising the administration of a therapeutically effective amount of an LP85 analog or LP85 functional fragment.

In another embodiment, the present invention relates to a method for treating wounds or bone fractures comprising the administration of a therapeutically effective amount of an LP85 analog or LP85 functional fragment.

In another embodiment, the present invention relates to a method for treating periodontal diseases comprising the administration of a therapeutically effective amount of an LP85 analog or LP85 functional fragment.

For therapeutic utility, an effective amount of an LP85 analog or LP85 functional fragment is administered to an organism in need thereof in a dose between about 0.1 and 1000 μg/kg body weight. In practicing the methods contemplated by this invention, the LP85 polypeptides or LP85 analogs of the present invention can be administered in multiple doses per day, in single daily doses, in weekly doses, or at any other regular interval. The amount per administration and frequency of administration will be determined by a physician and depend on such factors as the nature and severity of the disease, and the age and general health of the patient.

Accordingly, patients at risk of bone deterioration may be given a regular dose of the compounds of the present invention, to prevent bone deterioration. Patients at greatest risk for bone deterioration are post-menopausal women and men above the age of 60. By “normal bone density” is meant within two standard deviations of the mean value for race, age and sex.

The present invention also provides a pharmaceutical composition comprising as the active agent an LP85 fragment, LP85 analog, and/or a pharmaceutically acceptable non-toxic salt thereof, and a pharmaceutically acceptable solid or liquid carrier. For example, compounds comprising at least one LP85 analog can be admixed with conventional pharmaceutical carriers and excipients, and used in the form of tablets, capsules, elixirs, suspensions, syrups, wafers, parenteral formulations, and the like. The compositions comprising at least one LP85 fragment and/or an LP85 analog, will contain from about 0.1% to 90% by weight of the active compound, and more generally from about 10% to 30%. The compositions may contain common carriers and excipients such as corn-starch or gelatin, lactose, sucrose, microcrystalline cellulose, kaolin, mannitol, dicalcium phosphate, sodium chloride, and alginic acid.

As a general proposition, the total pharmaceutically effective amount of the LP85 fragments or LP85 analogs of the present invention administered parenterally per dose will be in the range of about 1 μg/kg/day to 10 mg/kg/day of patient body weight, particularly 2 mg/kg/day to 8 mg/kg/day, more particularly 2 mg/kg/day to 4 mg/kg/day, even more particularly 2.2 mg/kg/day to 3.3 mg/kg/day, and finally 2.5 mg/kg/day, although, as noted above, this will be subject to therapeutic discretion. More preferably, this dose is at least 0.01 mg/kg/day. If given continuously, the LP85 analogs of the present invention are typically administered at a dose rate of about 1 pg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the LP85 analogs of the present invention may be administered orally, rectally, intracranially, parenterally, intracisternally, intravaginally, intraperitoneally, topically (as by powders, ointments, drops or transdermal patch), transdermally, intrathecally, bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein includes, but is not limited to, modes of administration which include intravenous, intramuscular, intraperitoneal, intrasternal, subcutaneous and intraarticular injection, infusion and implants comprising LP85 analogs.

The compounds can be formulated for oral or parenteral administration. A preferred parenteral formulation for subcutaneous administration would comprise a buffer (phosphate, citrate, acetate, borate, TRIS), salt (NaCl, KCl), divalent metal (Zn, Ca), and isotonicty agent (glycerol, mannitol), detergent (Polyoxyethylene sorbitan fatyy acid esters, poloxamer, ddicusate sodium, sodium lauryl sulfate), antioxidants (ascorbic acid), and antimicrobial agent (phenol, m-cresol, alcohol, benzyl alcohol, butylparben, methylparaben, ethylparaben, chlorocresol, phenoxyethanol, phenylethyl alcohol, propylparaben.

For intravenous (IV) use, the LP85 analog is administered in commonly used intravenous fluid(s) and administered by infusion. Such fluids, for example, physiological saline, Ringer's solution or 5% dextrose solution can be used.

For intramuscular preparations, a sterile formulation, preferably a suitable soluble salt form of LP85 analog such as the hydrochloride salt, can be dissolved and administered in a pharmaceutical diluent such as pyrogen-free water (distilled), physiological saline or 5% glucose solution. A suitable insoluble form of the compound may be prepared and administered as a suspension in an aqueous base or a pharmaceutically acceptable oil base, e.g. an ester of a long chain fatty acid such as ethyl oleate.

The LP85 analogs of the present invention are also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Other sustained-release compositions also include liposomally entrapped modified LP85 analogs. Such liposomes are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EDP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, in one embodiment, the LP85 analogs of the present invention are formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the LP85 analogs of the present invention uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The LP85 analogs of the present invention are typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of salts of the LP85 analogs of the present invention.

Polypeptides to be used for therapeutic administration must be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

LP85 analogs ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution of one of the LP85 analogs of the present invention, and the resulting mixture is lyophilized. The infusion solution is prepared by reconstituting the lyophilized polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the LP85 analogs of the present invention may be employed in conjunction with other therapeutical compounds.

The following examples more fully describe the present invention. Those skilled in the art will recognize that the particular reagents, equipment, and procedures described are merely illustrative and are not intended to limit the present invention in any manner.

### EXAMPLE 1

In Situ Hybridization and RT-PCR Amplification of LP85 cDNA

A panel of human tissue sections were screened as follows by in situ staining. After de-paraffinization, slides were pretreated with 10 μg/ml Proteinase K in PBS for 10 min. at room temp. and then washed in 0.1M glycine followed by 2×SSC. Slides were treated with acetic anhydride in 0.1 M TEA for 10 min. at room temp followed by a quick H₂O wash and then allowed to dry.

Fluorescein labeled riboprobes (sense and antisense) were generated using the DNA template from the PCR reaction in Example 4. T7 RNA polymerase promoters were added to the LP85 CDNA in a chosen orientation using “Lig'nScribe” from Ambion. Labelled RNA was generated from this template using an RNA Labelling Kit (117025) and fluoroscein label (1685619) from Boehringer Mannheim. Riboprobes were diluted in Dako hybridization buffer and added to the slides. The slides were coverslipped and incubated at 45° C. overnight. Hybridization was carried out in a humidified thermal cycler (Hybaid Omnislide). After hybridization, cover-slips were soaked off in 0.1%SDS in 2×SSC at room temp. A stringency wash of 0.1%SDS in 0.1×SSC was performed at 50° C. for 15 min. After thorough washing with 2×SSC, followed by a water wash, the slides were then coverslipped and viewed with a fluorescent microscope. LP85 was expressed strongly in the epithelium (cervix, vagina, tonsil), kidney, liver, placenta and gut in human tissue. Strong staining was also observed in osteoblasts from fetal baboon.

A LP85 cDNA was isolated by reverse transcriptase PCR (RT-PCR) using conventional methods. Briefly, total RNA from a tissue that expresses the LP85 mRNA, for example baboon femurs, is prepared using standard methods. First strand cDNA synthesis is achieved using a commercially available kit (SuperScript™ System; Life Technologies) in conjunction with specific primers directed at any suitable region of SEQ ID NO:1 between nucleotides 114 and 1223.

Amplification is carried out by adding to the first strand cDNA (dried under vacuum): 8 μl of 10×synthesis buffer (200 mM Tris-HCl, pH 8.4; 500 mM KCl, 25 mM MgCl₂, 1 ug/ul BSA); 68 μl distilled water; 1 μl each of a 10 uM solution of each primer; and 1 μl Taq DNA polymerase (2 to 5 U/μl). The reaction is heated at 94° C. for 5 min. to denature the RNA/cDNA hybrid. Then, 15 to 30 cycles of PCR amplification are performed using any suitable thermal cycle apparatus. The amplified sample may be analyzed by agarose gel electrophoresis to check for an appropriately-sized fragment. The amplified sample may be analyzed by agarose gel electrophoresis to check the length of the amplified fragment. Wild-type LP85 CDNA generated in this manner is then used as a template for introduction of point mutations (i.e. construction of LP85 analogs). The basic protocol described in details in “Current Protocols in Molecular Biology”, volume 1, section 8.5.7 (John Wiley and Sons, Inc. publishers), the entire teachings of which are incorporated herein by reference. In this protocol, synthetic oligonucleotides are designed to incorporate a point mutation at one end of an amplified fragment. Following first PCR, the amplified fragments encompassing the mutation are annealed with each other and extended by mutually primed synthesis. Annealing is followed by a second PCR step utilizing 5′ forward and 3′reverse end primers in which the entire mutagenized fragment gets amplified and is ready for subcloning into the appropriate vector.

### EXAMPLE 2

Construction of a Vector for Expressing LP85 analogs in a Host Cell

An expression vector suitable for expressing a LP85 analog in a variety of prokaryotic host cells, such as E. coli is easily made by one skilled in the art. The vector generally will contain an origin of replication (Ori), an ampicillin resistance gene (Amp) useful for selecting cells which have incorporated the vector following a tranformation procedure, and further comprise the T7 promoter and T7 terminator sequences in operable linkage to a LP85 coding region. Plasmid pET28A (obtained from Novogen, Madison Wis.) is a suitable parent plasmid. PET28A is linearized by restriction with endonucleases NdeI and BamHI and ligated to a LP85 fragment or LP85 analog encoding DNA fragment.

The LP85 fragment or LP85 analog encoding cDNA used in such constructions may be further modified at the 5′ end (amino terminus of encoded protein) in order to simplify purification of the encoded protein product subsequently expressed. For this purpose, an oligonucleotide encoding 6 histidine residues is inserted after the ATG start codon. Placement of the histidine residues at the amino terminus of the encoded protein serves to enable the IMAC one-step protein purification procedure described previously.

Three different LP85 constructs were ligated into the Not I/XbaI cloning site of a CMV-Flag vector having a preprotrypsin signal peptide for forced secretion in mammalian cells followed by an N-terminal FLAG tag (DYKDDDDK) epitope. As a result of cloning procedure, there are 4 irrelevant amino acids (LAAA) between the Flag Tag and the LP85 amino acid sequence of all constructs. LP85-N-10 was generated by PCR using 5′AATGGAAAAAAGCGGCCGCGATCTGCGCAAACTTTTGCAGCTCTC-3′ with a Not I site as the forward primer3 ′ACCAGCTCTAGATTATCGAGGGTGGTCTTGAGCTGC-5′ having an XbaI site. LP85-N-3C is identical to LP85-N-10 except that the 3 N-terminal cysteines at amino acid 12, 16 and 18 (as shown in SEQ ID NO:2) are replaced with serine as the result of being generating the cDNA via amplification with the following primers: PCR forward primer sequence 5′-GCTTGCGGCCGCGATCTCCGCAAACTT TCCAGCTCTCGGGACACTTCTGCAACCCCGCA-3′ with NotI site Reverse primer sequence: 3′-ACCAGCTCTAGATTATCGAGGGTGGTCTTG AGCTGC-5′ with XbaI site. LP85-N-18 represents a polypeptide comprising the mature form of LP85 (i.e., amino acid residues 19-370 as shown in SEQ ID NO:2) and was generated with PCR forward primer sequence 5′-GCTTGCGGCCGCGCGGGACACTTCTGCAACCCCGCAG-3′ with Not I site and reverse primer sequence 3′-ACCAGCTCTAGATTA TCGAGGGTGGTCTTGAGCTGC with XbaI site.

LP85R233Q represents a polypeptide comprising the mature form of LP85 (19-370 amino acid as shown in SEQ ID NO:2) with the Arg at position 249 replaced with Gln. Introduction of the LP85R233Q mutation was done by PCR using a SOEing (Sequential Overlapping Extension) reaction. The first PCR reaction fragment was generated by the forward primer 5′-gacaagcttcgggacacttctgcaaccccgcagagcgcatccatcaaagcattgcg and reverse primer 5′-cttttgacttccggtcatggtatgattggcctcgataccg to yield a 727 base pair fragment. A second PCR reaction fragment was generated by the forward primer 5′-ggtatcgaggccaatcataccatgaccggaagtcaaaag and the reverse primer 5′-gcgggatcctctagattatcagccggctcgaggtggtcttgagctgcagatacaatc to yield a 395 base pair fragment. A standard PCR reaction was performed to generate both products (1 cycle of 95° C.-5 minutes; 25 cycles of 95° C.-30seconds, 58° C.-30 seconds, 72° C.-1 minute and 1 cycle of 72° C.-7 minutes). Fragments from PCR 1 and PCR 2 were mixed 1:1 and used as the template for a final PCR reaction. Using the forward primer 5′-gacaagcttcgggacacttctgcaaccccgcagagcgcatccatcaaagcattgcg and the reverse primer 5′-gcgggatcctctagattatcagccggctcgaggtggtcttgagctgcagatacaatc, a fragment of 1083 base pairs was isolated. The fragment was generated using the following PCR conditions 1 cycle of 95° C.-5 minutes; 5 cycles of 95° C.-30 seconds, 50° C.-30 seconds, 72° C.-1 minute; 20 cycles of 95° C.-30 seconds, 58° C.-30 seconds, 72° C.-1 minute and 1 cycle of 72° C.-7 minutes. Following sequence confirmation, a 580 base pair EcoRV-Eco47III fragment was subcloned into the original LP85N-18 expression construct.

### EXAMPLE 3

Recombinant Expression and Purification of Bioactive LP85 and Analogs

A. Expression in E. coli

An overnight culture of at least one of the E. coli transformants obtained from methods in Example 2 was diluted 1:40 into fresh 2×TY broth containing 25 μg/ml kanamycin and incubated for approximately 90 minutes at 37° C. Recombinant protein expression was then induced by the addition of 0.5 mM IPTG. After approximately six hours at 37° C., the cells were isolated from the media by centrifugation and then lysed using a combination of DNAse I, lysozyme, and sonication. The granules of insoluble inclusion bodies of LP85 protein released upon lysis were washed with Tris buffered EDTA, Tris buffered EDTA containing 0.5M KCL and MilliQ water.

The isolated granules were solubilized in 7M urea, 50 mM Tris, 10 mM dithiothreitol, pH 8, and diluted 10-fold into a solution of 7M urea, 50 mM Tris, 10 mM cyseine HCl, pH 8. After stirring at room temperature for approximately one hour, the solution was placed into 3500 MWCO dialysis tubing and dialyzed against three changes of PNAG buffer (nine parts 1×PBS, 0.35 M NaCl, 0.5 M Arginine HCl, pH 8 and one part glycerol) at about 8° C. (cold room). The final concentration was about 0.1 mg/ml in the dialysis bag. The resultant fold was purified by size exclusion chromatograpphy on a 6×60 cm Superdex 75 column equilibrated in PHAG buffer and eluted at a flow rate of about 10 ml/min at room temperature, and 20 ml fractions were collected. Variously-sized LP85 polypeptide moieties (as determined by non-reducing SDS-PAGE) were pooled and tested for bio-activity.

B. Expression in Mammalian Cells

LP85 fragment or LP85 analog encoding constructs described in Example 2 were transfected into 293 cells for mammalian protein expression. HEK293T and HEK293E cells were plated in 225 cm flasks and grown to about 70% confluence. A transfection mixture of 30 μg LP85 cDNA vector and 100 μg Fugene in 2 ml of warm OPTI MEM I medium was mixed and pre-incubated at room temperature for 15 minutes before adding to cells. Cells were washed once with lx PBS and 25 ml of 1% FBS DMEM/F12 medium and the LP85-Fugene complex was added to each 225 cm flask. These flasks were incubated at 37° C. with 5% C02. Culture conditioned medium was collected at 72 hr post-transfection. 25 ml of fresh 1% FBS DMEM/F12 medium were added to each flask and conditioned media was collected again at day 6 post-transfection. Transfectants were analyzed for transient expression of LP85 using Western blot analysis. The results showed that the LP85 protein was secreted into the culture medium by cells transformed with all constructs.

Three different LP85 constructs were transiently expressed in HEK293T cell line with a FLAG tag (DYKDDDDK) at the N-terminus. An antibody-affinity procedure was used in purification of flag peptide tagged LP85-N-10 (WT), LP85-C3-N-10 and LP85-N-18. Briefly, the culture medium was passed through 0.22 micron filter. The culture filtrate was mixed batch-wise with the anti-flag M2-agarose affinity gel (Sigma; St. Louis, Mo.) and shaken gently overnight at 4-C. The gel was collected by a suitable column and washed extensively with PBS with 0.4M NaCl (Buffer A). The protein was eluted with 100 micromolar flag peptide (Sigma; St. Louis, Mo.) in Buffer A. Each LP85 polypeptide was 95-100% purified by this antibody-affinity procedure, as shown by SDS-PAGE of the two mutated LP85 proteins as described below.

The purified LP85 protein samples were run on SDS-PAGE gel under non-reducing and reducing conditions. Under non-reducing conditions, from the N-terminal sequencing analysis, the major band around 90-kDa was identified as covalently linked full-length (“FL”) dimer and was shifted to around 50-kDa (as FL-monomer) upon reduction with β-mercaptoethanol The broad band indicates potential glycosylation of the protein. The 12-residue amino-terminal sequence of the protein around 32-kDa is SYHDRKSKVDLD, confirming this protein as covalently linked carboxy (“C”)-terminal dimer, resulting from a tryptic or trypsin-like cleavage of FL-dimer at Arg249 corresponding to SEQ ID: No 2 during the expression and/or the purification. This band shifted to about 16-kDa upon reduction. The corresponding amino (“N”)-terminal cleavage product was shown as (possibly glycosylated) 42-kDa monomer. The amino acid sequence for the N-terminal fragment does not contain consensus sequence (Asn-Xaa-Thr/Ser) for N-linked glycosylation, therefore the glycosylation is likely to result from O-linked glycosylation at Thr or Ser. In addition, the 60-kDa protein was attributed to N/C dimer (with slightly higher than 1:1 molar ratio), in which one of the two monomer was cleaved. The high MW (greater than 200-kDa) LP85-C3-N-10 proteins were minor species (less than 10%), in contrast to predominant species (greater than 80%) for LP85-WT (not shown).

A similar pattern was observed for LP85-N-18 deletion mutant protein on SDS-PAGE gel as shown below except for the absence of any high MW protein from LP85-N-18 (under non-reducing conditions) and also for the absence of any minor degradation products (under reducing conditions).

In addition, the purified samples have been analyzed by size-exclusion HPLC under native conditions (PBS+0.4 M NaCl, pH 7.8). A 25 microliter sample, containing about 0.5-0.6 mg/ml protein in PBS+0.4 M NaCl, pH 7.8, was loaded on to a TSK-G3000swl column (TOSOHAAS). Both LP85 proteins ran about 100 to 200-kDa with a very small amount of aggregates of greater than 600-kDa. The MW of LP85-C3-N-10 and LP85-N-18 was determined to be around 100-kDa by analytical ultracentrifugation analysis, indicating that both of these proteins are dimeric under native conditions.

The protease resistant mutant containing a FLAG tag was purified from culture media following concentration in an Amicon ProFlux M12 tangential filtration system to about 500 ml using an Amicon S3Y10 UF membrane. Protease inhibitors (Roche, inhibitor cocktail tablets, cat. #1873580) were added (1 tablet per liter of media). The concentrated media was incubated overnight with Anti-FLAG M2-Agarose affinity column (Sigma [A-1205], 10 to 25 ml of resin). The resin was then spun down in a Jouan table-top centrifuge, CR412, at 1000 rpm for 5 min. The resin was then packed into a column and washed with buffer A at a flow rate of 3 ml/min (20 mM Tris, 150 mM NaCl, pH 7.4) until the absorbence returned to baseline, and the bound polypeptides were eluted with 100 ug/ml FLAG peptide (Sigma) (in buffer A). Fractions containing the LP85 analog were pooled and concentrated using an Ultrafree centrifugal filter unit (Millipore, 10 kDa molecular weight cut-off) to 20 ml. This material was passed over a Superdex 200 (Pharmacia, 35/600) sizing column equilibrated with PBS, 0.5 M NaCl, pH 7.4, at a flow rate of 5.0 ml/min. Fractions containing the LP85R233Q were analyzed by SDS-PAGE. The N-terminal sequence of the LP85R233Q was confirmed on the purified polypeptide. In contrast to wt LP85, LP85R233Q showed a single band around 50 kDa under reducing conditions. This mutant prevents proteolytic cleavage observed for wt LP85.

### EXAMPLE 4

Detecting Ligands that Bind LP85 Using a Chaperonin Protein Assay

The wells of an ELISA plate are coated with chaperonin by incubation for several hours with a 4 μg/ml solution of the protein in Tris-buffered Saline (TBS: 10 mM Tris-HCl, pH 7.5, 0.2 M NaCl). The plates are then washed 3 times with TBS containing 0.1% Tween-20 (TBST). Then, a mixture of LP85 protein (sufficient amount to saturate about 50% of the binding sites on chaperonin) and test compound (10⁻⁹ to 10⁻⁵ M) in about 50 μl volume is added to each well of the plate for an incubation of about 60 minutes. Aliquots of the well solutions are then transferred to the wells of fresh plates and incubated for 60 minutes at room temperature, followed by 3 washes with TBST. Next, about 50 μl of an antibody specific for LP85 plus 5% nonfat dry milk are added to each well for a 30 minute incubation at room temperature. After washing, about 50 μl of goat anti-rabbit IgG alkaline phosphatase conjugate at an appropriate dilution in TBST plus 5% nonfat dry milk are added to each will and incubated 30 minutes at room temperature. The plates are washed again with TBST and 0.1 ml of 1 mg/ml p-nitrophenylphosphate in 0.1% diethanolamine is added. Color development (proportional to bound alkaline phosphatase antibody conjugate) is monitored with an ELISA plate reader. When test ligand binding has occurred, ELISA analysis reveals LP85 in solution at higher concentrations than in the absence of test ligand.

### EXAMPLE 5

Use of LP85 Fragments or LP85 Analogs to Treat Osteoporosis

Experimental models of postmenopausal osteoporosis are known in the art. Germane to this invention is the ovariectomized rat model which is provided in U.S. Pat. No. 5,393,763 which is incorporated herein by reference.

An additional demonstration of the method of treating or preventing osteoporosis due to estrogen deprivation would be as follows: One hundred patients would be chosen, who are healthy postmenopausal women, aged 45-60 and who would normally be considered candidates for estrogen replacement therapy. This includes women with an intact uterus, who have had a last menstrual period more than six months, but less than six years. Patients excluded for the study would be those who have taken estrogens, progestins, or corticosteroids six months prior to the study or who have ever taken bis-phosphonates.

Fifty women (test group) would receive a pharmaceutical composition comprising at least one LP85 analog. The other fifty women (control group) would receive a matched placebo per day. Both groups would receive calcium carbonate tablets (648 mg) per day.

A baseline examination of each patient includes quantitative measurement of urinary calcium, creatinine, hydroxyproline, and pyridinoline crosslinks. Blood samples are measured for serum levels of osteocalcin and bone-specific alkaline phosphatase. Baseline measurements would also include a uterine examination and bone mineral density determination by photon absorptiometry.

The study would continue for at least six months, and each patient would be examined for changes in the above parameters. During the course of treatment, the patients in the treatment group would show a decreased change in the biochemical markers of bone resorption as compared to the control group. Also, the treatment group would show little or no decrease in bone mineral density compared to the control group. Both groups would have similar uterine histology.

### EXAMPLE 6

Endothelial Cell Growth Assay

To assay the effects of LP85 analogs on endothelial cell growth, approximately 3000 human umbilical vein endothelial cells were grown in 96 well TC plates in 150 μl Media 199/10% fetal calf serum with and without LP85. LP85 proteins were added to a final concentration of 0.1 ng/ml-1 μg/ml.

After 24 hours post-plating, approximately 0.25 μCi ³H-thymidine was added to each well. Forty-eight hours later, plates were frozen at −70° C., thawed, cells harvested onto filter paper and the samples counted in a scintillation counter.

### EXAMPLE 7

LP85 Exposure to Cell Proliferation Panel

The following cell panels may be exposed to LP85 analogs, to assay their effects on cell proliferation:

CTLL.6 (Murine)

Cytotoxic T-cell line, from C57/Bl/6 mice, lymphoblast morphology, IL-2 dependent

MTS proliferation assays

Growth Medium: RPMI 1640/L-Glu+10%FBS+1 mM Sodium

Pyruvate+10 mM HEPES+5×10⁻⁵ M 2-Me+2 ng/ml IL-2

Assay Medium: RPMI 1640/L-Glu+10%FBS+1 mM Sodium Pyruvate+10 mM HEPES+5×10⁻⁵ M 2-Me.

T1165.17 (Murine)

Plasmacytoma cell line (originates from B-cell), from ascites tumors of Balb/CanPt mice

IL-1 dependent

MTS proliferation assay

Growth Medium: RPMI 1640/L-Glu+10%FBS+1 mM Sodium Pyruvate+10 mM HEPES+5×10⁻⁵ M 2-Me+2 ng/ml rhIL-1

Assay Medium: RPMI 1640/L-Glu+10%FBS+1 mM Sodium Pyruvate+10 mM HEPES+5×10⁻⁵ M 2-Me

BalbC/3T3 Clone A31 (Murine)

From 14-17-day-old Balb/C mouse embryos, fibroblast morphology, non-tumorigenic, contact-inhibited ³H Thymidine uptake proliferation assays

Growth Medium: DMEM+10% Calf Serum

Assay medium: DMEM+2% Plasma Dialyzed Calf Serum

TF.1 (Human)

Erythroleukemia, lymphoblast morphology

hGM-CSF dependent

MTS proliferation assays

Growth Medium: RPMI 1640/L-Glu+10%FBS+1 mM Sodium Pyruvate+10 mM HEPES+5×10⁻⁵ M 2-Me+5 ng/ml rhGM-CSF

Assay Medium: RPMI 1640/L-Glu+10%FBS+1 mM Sodium

Pyruvate+10 mM HEPES+5×10⁻⁵ M 2-Me

MCF-7

Human breast cancer cell line, epithelial morphology

³H Thymidine uptake proliferation assays

Responds by growth to insulin

Growth Medium: Eagles MEM (w/o phenol red)+10% FBS+1 mM Sodium Pyruvate+Nonessential Amino Acids+L-Glu+1 μg/ml insulin

Assay Medium: Eagles MEM (w/o phenol red)+1 mM Sodium Pyruvate+Nonessential Amino Acids+L-Glu+10 μg/ml human transferrin

HUVEC

Human umbilical vein endothelial cells, primary

³H Thymidine uptake proliferation assays

Growth Medium: Clonetics complete endothelial growth medium

Assay Medium: Medium 199+10% FBS

In BalbC/3T3 proliferation experiments approximately 5000 BalbC/3T3 cells were seeded per well in a 96 well plate in DMEM/10% calf serum. The cells were grown for two days in an incubator to achieve approximately 90-100% confluence. Then, cells were starved in DMEM/2% dialyzed calf serum for 24 hours in the incubator and growth factors were added diluted in DMEM/2% dialyzed calf serum. The final volume per well was 200 μl. Cells were incubated an additional 16-18 hours.

To assess the effects of LP85 proteins and other growth factors on cell proliferation, each well received 0.25 uCi ³H-thymidine during a 2 hour pulse in the incubator. Cells were then harvested and counted in a scintillation counter. Human PDGF, isolated from human platelets, was purchased from R & D Systems (cat # 120-HD-001). All positive controls (i.e. human PDGF) showed the activity expected.

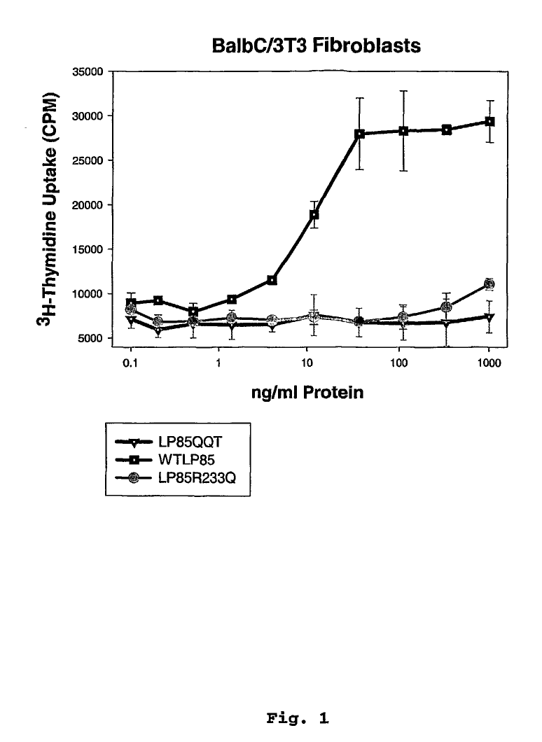

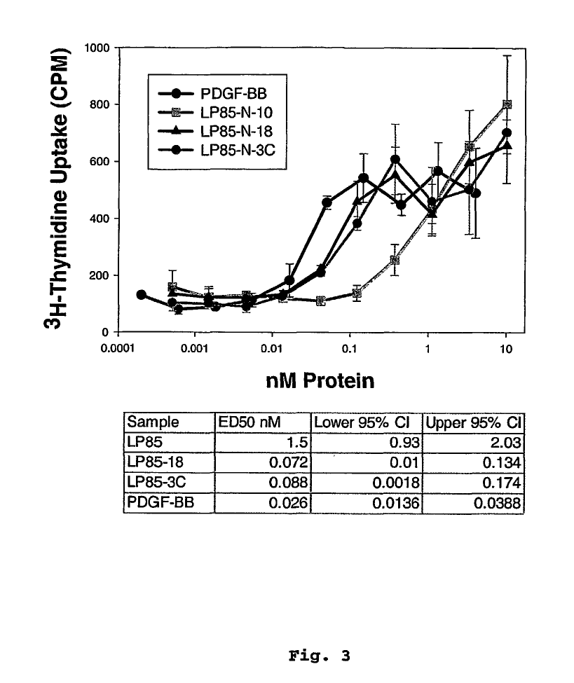

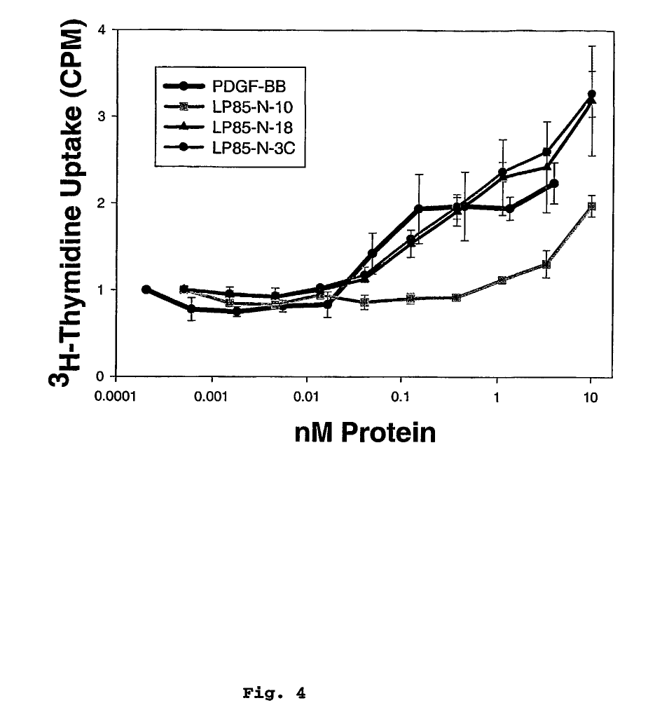

FIG. 1 shows that various LP85 proteins can measurably stimulate thymidine uptake in BalbC/3T3 fibroblast cells when added at 10-1000 ng/ml protein. FIG. 3 and FIG. 4 shows similar effect of LP85 on human dermal fibroblasts and rat L6 skeletal muscle cells, respectively.

### EXAMPLE 8

Assay for LP85 Antagonist

Assay reactions are set up essentially as described in Example 7, except that a compound to be tested for LP85 antagonist activity is included at the step of adding conditioned medium to bovine capillary endothelial cells. Multiple assays can be set up in which a constant amount of conditioned medium is incubated with varying amounts of test compound, for example from about 10 ng/ml to about 100 μg/ml.

For conducting the cell growth assay, bovine capillary endothelial cells are maintained in DMEM containing 20% calf serum according to the method of Ferrara, Biochem. Biophys. Res. Comm., 161, 851-58, 1989. Cells are plated at about 8×10³ cells per well in 12 well plates in DMEM supplemented with 10% calf serum, 2 mM glutamine, and antibiotics. Conditioned medium from transiently transfected 293 cells 72 hours post-transfection is added and cell number determined after 5 days.

### EXAMPLE 9

LP85 Stimulates Human Aortic Smooth Muscle Cells

Approximately 5000 human aortic smooth muscle cells are seeded per well in a 96 well plate containing smooth muscle growth media (SMGM) from Clonetics. Cells are grown overnight in an incubator. After overnight incubation, SMGM was replaced with 100 μl/well smooth muscle basal media (SMBM). Cells are starved in SMGM for 48 hours in the incubator. Next, LP85 fragments or LP85 analogs diluted in SMBM are added to each well. Human PDGF, isolated from human platelets (R&D Systems (cat. #120-HD-001) was used as a positive control. The final volume per well is approximately 200 μl and cells are incubated for approximately 20 hours.

To assess the effect on cell proliferation approximately 0.25 μCi ³H-thymidine is added to each well for 4 hours in the incubator. Cells are harvested and the quantity of radioactivity taken up by the cells was determined using a scintillation counter (data not shown).

### EXAMPLE 10

Activation of MAP Kinase by LP85 Analogs

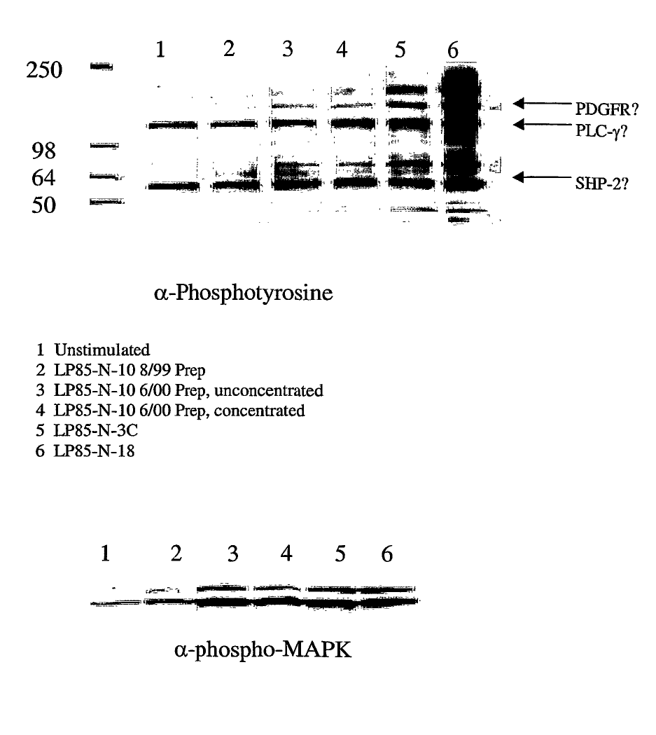

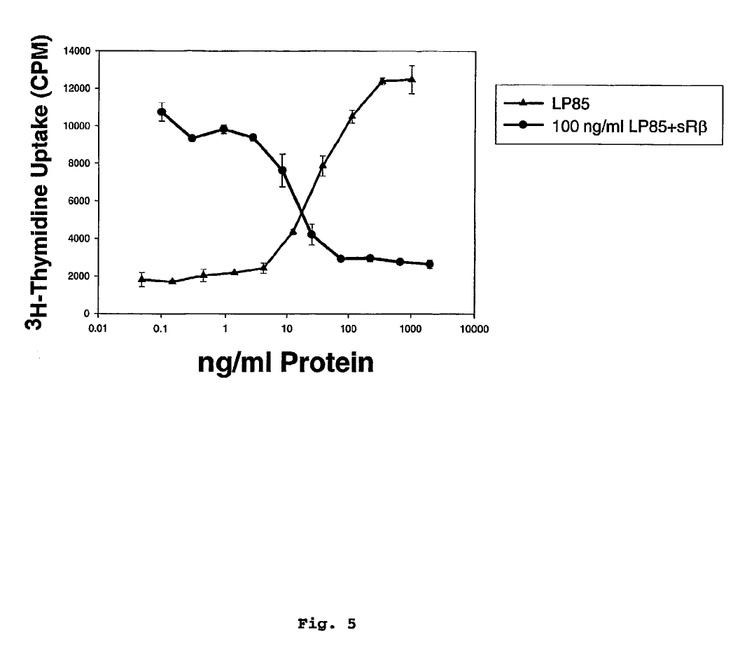

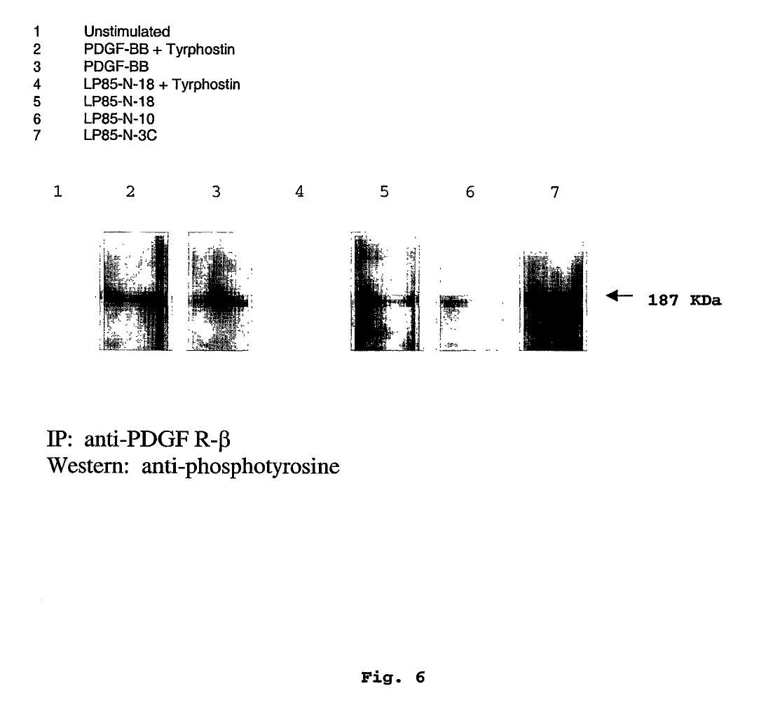

Fifty thousand BalbC/3T3 cells were plated per well in 24 well dishes in DMEM/10% calf serum and incubated overnight at 37° C. and 5% CO₂. The following day when the cells were about 80% confluent, the medium was replaced with DMEM-no serum and the cells were serum starved for about seven hours at 37° C. Cells were then stimulated with 1 μg/ml LP85 in 400 μl DMEM without serum for 10 minutes at room temperature. After stimulation, the ligand containing medium was aspirated and about 100 μg of lysis buffer (50 mM Hepes, pH 7.5, 150 mM NaCl, 10% glycerol, 1% Triton X-100, 1 mM EDTA, 1 mM pervanadate) was added to the cells. After lysis, lysates were cleared by spinning through Millipore Ultrafree-MC filters (UFC30HVNB) at 12,000 RPM for 1 minute. About 15 μl of the lysate was analyzed on a reducing 8-16% Tris-glycine SDS-PAGE gel. After electrophoresis the samples were transferred to a nitrocellulose membrane. Membranes were blocked for 1 hr. with TBS with 0.1% Tween and 5% BSA. Western blot analysis was performed using phospho-specific antibodies Phospho-p44/42 MAPK (NEB#9191L; New England Biolabs (NEB)) and anti-phosphotyrosine (Upstate #05-321)(see FIG. 2). Alternatively, lysates were first immunoprecipitated using anti-PDGFRP antibodies (NEB) and then subjected to Western blot analysis using phosphotyrosine antibodies (NEB); see FIG. 2). Generally, PDGFRβ activation leads to tyrosine phosphorylation of several intracellular substrates including but not limited to PLC-Y and SHP-2 and eventual activation of MAP kinases (ERK1 and ERK2). LP85 stimulation of BalbC/3T3 cells mimics the effects of the PDGFRβ receptor-mediated activation whereby PDGFRP becomes tyrosine phosphorylated and MapK is activated (FIG. 2 and FIG. 6). Furthermore, preincubating LP85 with soluble PDGFR-P completely antagonizes the mitogenic effect of LP85 fragments or LP85 analogs (FIG. 5).

### EXAMPLE 11

Effect of LP85 Analogs on Osteocalcin Promoter Activation

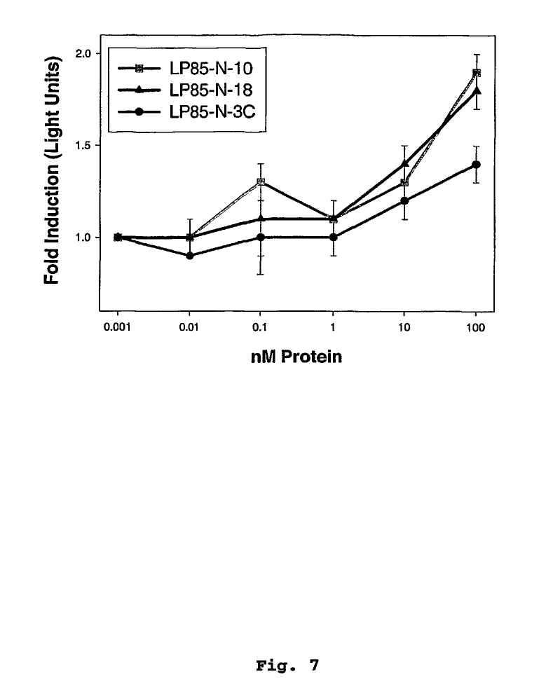

Rat Osteosarcoma cells (ROS) which stabily express the osteocalcin (OCN) promoter driving a luciferase reporter gene were seeded in a white walled/clear bottom 96-well plate at 50,000 cells/well in DMEM/10% FCS. The following day, the media was replaced with DMEM/0.1% FCS and cells were incubated overnight at 37° C./5% CO₂. The following day, the starving media was removed and the cells were stimulated with LP85 protein in DMEM/0.1% FCS for 6-8 hours at 37° C. After stimulation, media was aspirated and cells were rinsed in PBS. Cells were then lysed in 50 μl luciferase assay buffer (1 mM MgCl2/150 μM ATP/5 mM DTT/0.1% Triton X-100/0.5 mM Luciferin/100 gm CoA/50 mM Tris) and luciferase activity was measured with a luminometer (FIG. 7).

### EXAMPLE 12

LP85 Stimulates Rat Metatarsal Bone Growth In Vitro

PDGF has been shown to increase bone mass and close cartilage growth plates in the long bones of rats, and to stimulate fibrosis in connective tissues, in vivo (B. Mitlak et al. J. Bone & Miner. Res., 11:238-247, (1996)). Like PTH, a known anabolic agent to increase skeletal mass, PDGF also stimulates interstitial collagenase, a step thought to be necessary to stimulate bone turnover, thereby increasing the proportion of new matrix. In vitro, PDGF stimulates proliferation of osteoprogenitor cells in fetal rat calvaria, and the rate of bone collagen synthesis (See e.g. Hock, J. and Canalis, E., Endrocrin., 134, (1994)). PDGF also stimulates proliferation of muscle cells and chondrocytes, suggesting significant effects on the homeostasis of these tissues.

LP85 functions like other PDGFs to stimulate smooth muscle cells and fibroblast cell proliferation. Moreover, biochemical studies have shown that LP85 can activate the PDGF receptor (PDGF-R) as well as downstream signaling events such as MAP Kinase activation (see Example 11). These results suggest that LP85 may exert its biological influence through the PDGFR-β. Thus, LP85 can mimic PDGF function and play a role in cell proliferation, including muscle cell proliferation, wound healing, and bone growth.

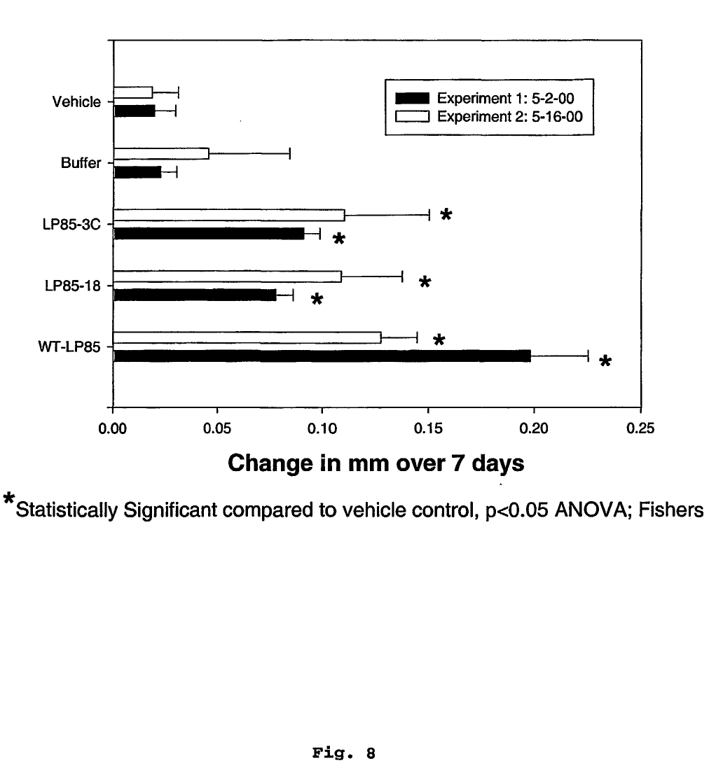

Newborn Sprague Dawley rats (Harlan, Indianapolis, Ind.) were sacrificed at day 0 and the metatarsals surgically removed and placed in BGJ medium (Life Technologies, Rockville, Md.) without serum and containing an antibiotic-antimycotic solution. The metatarsals were cultured for 7 days in the presence of vehicle, PDGF (100 ng/ml +/−Genistein 100 μM), or LP85N-10 and LP85N-18 (10 ng/ml and 100 ng/ml +/−Genistein 100 μM), in a 96 well round bottom petri dish under 5% CO₂ at 37° C. Genistein is a boad based tyrosine kinase inhibitor. The medium was changed every 24 hours during the 7 day treatment period. Metatarsals were imaged under a light microscope and changes in mineralization quantified using Image Pro™ analysis software package. Anabolic activity was quantified over the 7-day period as the increase in endochondral ossification measured as the longitudinal extension of the mineralized region (see FIG. 8).

### EXAMPLE 13

Use of LP85 Analogs to Treat Sarcopenia

The efficacy of LP85 in treating sarcopenia is evaluated in elderly males between the ages of 55-100. Test subjects are monitored at regular intervals for muscle mass, muscle weakness and increased fatigability, as described, for example, in Fiatarone et al., JAMA, 263:3029-3034, (1990), herein incorporated by reference. Medical histories including interviews with family members are taken. Based on the clinical tests, physical examinations, and medical histories subjects are identified as having sarcopenia for the test group. Once per day treatments with a LP85 protein are administered as an bolus of 2.5 mg/kg by any suitable route of administration. Treatment with the LP85 protein is continued from four days to at least four week. Test subjects are monitored throughout the test period for changes in muscle mass, muscle weakness and fatigability.

### EXAMPLE 14

LP85 Analogs Can Stimulate Rat Bone Formation and Skeletal Development

In the mouse, a spontaneous deletion of the PDGF-α receptor gene results in abnormolitis including maldevelopment of craniofacial bones and vertebrae (Smith et al. Proc Natl Acad Sci 88:4811-4815 (1991), Stephenson DA et al. Proc Natl Acad Sci 88:6-10, (1991)). PDGF-BB has been also showed to accelerate the healing of tibial osteotomices, increase bone strength across healing fractures and stimulate endosteal and periosteal bone formation in rabbits (Nash TJ et al. 15:203-208, 1994). In rat study, PDGF administration induced extraskeletal collagen deposition and increases bone density and strength. LP85 fragments and/or analogs thereof can be tested for such activities as described below.

Virgin, virus-antibody-free, rapid growing (2 months old) intact rats and skeletal mature, ovariectomy(Ovx)-induced osteopenic Sprague-Dawley rats (Harlan Sprague Dawley, Inc.) are selected for treatment and control groups (baseline control animals that are killed at the day 0 and age-matched control groups). Initial doses of LP85 fragments and/or analogs ranging from between approximately 0, 50, 500, 1000, 2000, up to approximately 5000 μg/kg/d) can be given to the rats intravenous injection through a jugular vein catheter for 3 and 12 weeks. PTH (1-38), a known bone anabolic agent can be given to a group of rats as the positive control. To label the onset of the newly formed bone during the treatment period, at the first day of starting treatment, all rats can be given xylenol orange 90 mg/kg to label newly formed bone. All rats can also be given democlocycline 20 mg/kg s.c. on days 9, 8 and calcein 10 mg/kg (Sigma, St. Louis, Mo.) on days 2, 1 before sacrifice for bone histomorphometric quantitation. At necropsy, bones can be removed, cleaned of soft tissue, fixed in 10% formalin for 48 hours, then stored at 4° C. in 70% ethanol for later analyses. In vivo whole body composition, bone mineral content can be examined by dual energy x-ray densitometry (DEXA) and ex vivo analyses of bone mineral density, bone mineral content can be conducted by pQCT and high resolution micro-CT (Norlan/Stratec, Fort Atkinson, Wis.). Bone histomorphometry can be performed to analyze longitudinal bone growth rate, cartilage, bone mass and structure, bone formation and resorption indices and mechanical test can be conducted to compare the bone strength. The effects of treatment with LP85 fragments and/or analogs of the present invention can assessed in regards to decreases in body fat mass, stimulation of bone and cartilage development, increases in bone mass and bone strength in both intact, young growing and mature, osteopenic rats. The treatment groups can also be assessed for increased bone formation and decreased bone resorption histologically and with respect to serum markers as compared to the vehicle group.

### EXAMPLE 15

Use of LP85 Analogs to Treat Osteoarthritis

A rat meniscal tear model of osteoarthritis can be used to determine intra-articular efficacy of LP85 protein in inducing repair of meniscal tear-induced chondrocyte death/cartilage degeneration 4 weeks post-surgery in rats with treatment occurring for 3 weeks (2×/week).

40 Male Lewis rats, weighing approx. 325 grams at start(10/group), housed 2/cage, will be anesthetized with Isoflurane and the right knee area prepared for surgery. A skin incision will be made over the medial aspect of the knee and the medial collateral ligament will be exposed by blunt dissection, and then transected. The medial meniscus will be reflected medially with a fine hemostat and a cut will be made through the full thickness to simulate a complete tear. The skin will be closed with suture. Dosing by the intra-articular route will be initiated 4 weeks after surgery to allow full repair of joint capsule and will be continued for 3 weeks with intra-articular injections given twice weekly.

At necropsy, the right (operated) knee joint will be trimmed of muscle and connective tissue and collected into 10% neutral buffered formalin. The patella will be removed to allow proper fixation of the joints.

Following 4-6 days in Surgipath Decal, the operated joints will be cut into 2 approximately equal halves in the frontal plane, embedded and sectioned. A second and 3rd set of sections will be cut approximately 200 μm into each block, and sections will be stained with toluidine blue thus giving a total of 3 toluidine blue sections/knee joint per operated knee. So the total for the study would be 60 toluidine blue sections for the OA model.

Medial femoral and medial tibial cartilage degeneration will be scored for severity of cartilage degeneration using the following system: Depth of chondrocyte and proteoglycan loss with fibrillation

1=minimal superficial zone only

2=mild extends into the upper middle zone

3=moderate well into the middle zone

4=marked into the deep zone but not to tidemark

5=severe full thickness degeneration to tidemark

Area involved will be ⅓, ⅔ or all of the surface, if ⅓, multiply depth score times 1, if ⅔ multiply times 2 and if 3 multiply times 3. This may be changed to ¼, 2/4, ¾ or 4/4×depth score if lesion warrants.

In addition, for the tibial degenerative change, a micrometer measurement will be taken across areas of degeneration that resulted in significant matrix loss (greater than 50% of the cartilage thickness) in an effort to further quantitate the more serious changes. Finally, a micrometer depth of any type of lesion or morphologic change (cell/proteoglycan loss, change in metachromasia, but may have good retention of collagenous matrix and no surface fibrillation) expressed as a ratio of depth of changed area vs. depth to tidemark is taken over 4 equally spaced points on the tibial surface. This gives the most critical analysis of any type of microscopic change present and includes changes in matrix subjacent to non fibrillated areas.

Scoring of the osteophytes and categorization into small, medium and large is done with an ocular micrometer. Data are graphed both as score (0-3) or actual measurement. Osteophyte Evaluation=1, 2, or 3 for small, medium or large depending on size

1=small up to 299 μm

2=moderate 300-399 μm

3=large 400 or greater μm

Synovial reaction will be described if abnormal (should be mainly fibrosis) and characterized with respect to inflammation type and degree but will not be included in the score.

Sclerosis of subchondral bone will be described if present and documented but not included in the overall score.

These numbers will be summed to arrive at a total joint score which is based on the most severe morphologic alterations in the various sections evaluated from each rat.

In addition to documenting the degenerative changes, descriptions will be given of type and quality of reparative processes induced by treatment

Treatment Groups

| Group | N | Treatment |
|---|---|---|
| 1 | 10 | Surgery + vehicle or irrelevant protein ia |
| 2 | 10 | Surgery + protein A ia |
| 3 | 10 | Surgery + protein B ia |
| 4 | 10 | Surgery + protein C ia |

| | |
|---|---|
| D-7 | Rats arrive, begin acclimation |
| D0 | Surgery, 40 rats, tx intra-articular |
| D28 | Tx ia |
| D31 | Tx ia |
| D34 | Tx ia |
| D37 | Tx ia |
| D41 | Tx ia |
| D44 | Tx ia |
| D48 | Terminate and harvest right (operated) knee, process |
| | for histopathology. |

### EXAMPLE 16

LP85 Promotes Chemotaxis of Human Dermal Fibroblasts

Chemotaxis assays were performed using Boyden chambers. Test proteins were diluted in DMEM, 0.2 mg/ml β-lacto-globulin, 25 mM Hepes, pH 7.4 (assay media). 210 μl of diluted proteins were added to the lower wells of modified Boyden chambers. 0.8μ PVP-free Nuclepore filters were pretreated in 100 μg/ml Type 1 collagen for at least 24 hours. Filters were air-dryed immediately prior to use and placed directly over the diluted proteins. Low passage human dermal fibroblast cells were trypsinized and resuspended in assay media at 2.5×10⁵ cells/ml. 800 μl of cells were then added to the upper Boyden chambers. Chambers were then incubated for 4 hours in a humidified chamber of 5% CO₂ at 37° C. Chemotaxis was quantitated by fixing and staining cells on the protein side of the filter with Difquik. The dye was then extracted in 200 μl of 0.1N HCl for 20 minutes. The absorbance of the eluent was measured at 600 nm.

### EXAMPLE 17

Pharmacokinetic Comparison of LP85 Analogs

Studies were conducted to evaluate the plasma pharmacokinetic profiles of LP85 and LP85QQT (glycosylation and protease resistant mutant). LP85 and LP85QQT were administered to male Sprague-Dawley rats either intravenously (IV) or subcutaneously (SC) as a single dose. Blood samples were collected in EDTA and centrifuged to harvest the plasma for analysis by a sandwich enzyme-linked immunosorbent assay using polyclonal anti-LP85 antibodies. Plasma concentrations were estimated from a standard curve of LP85 in rat plasma ranging from 25 ng/ml to 0.39 ng/ml.

LP85 was cleared rapidly from the circulation after IV administration. Plasma concentrations were below the detection limit of the assay 2-3 hours after administration. The elimination half-life for LP85 was approximately 30 minutes. There was a dose-linear increase in AUC in recurring studies. The molecule showed no bioavailability after SC administration. After IV administration of the glycosylation mutant, LP85QQT, plasma concentrations were detectable out to 24 hours post-administration. The elimination half-life for LP85QQT was 3.5 hours, with an average clearance rate of 0.27 L/hr/kg. This clearance rate is approximately 4 times slower than observed with LP85 and is most likely attributed to the lack of clearance via hepatic mannose receptors.

### EXAMPLE 18

Effect of LP85 on Proteoglycan Synthesis by Articular Chondrocytes

The effect of LP85 or LP85 analogs on proteoglycan synthesis by articular chondrocytes may be evaluated as follows. Rabbit knee joints are collected and immersed in Dulbecco's PBS+pen-strep (D-PBS). Patella, muscle, and excess connective tissue is removed, the joints are disarticulated and are placed in a clean beaker of D-PBS. All extraneous tissue is carefully removed from the articular area, including synovium, tendon, ligament, fibrocartilage, etc., and the bones are again placed in a clean beaker of D-PBS. The articular cartilage from the medial and lateral tibial plateaus, from the medial and lateral femoral condyles, and from the patellar grove is shaved into a glass petri dish containing Ca²⁺, Mg²⁺-free D-PBS+pen-strep (CMF-PBS). The shaved cartilage is chopped into ˜1 mm² pieces with a scalpel. The chopped cartilage is collected into a 50 mL conical tube and digested in a 37° C. water bath sequentially with:

2 mg/ml hyaluronidase (Sigma H-2251, 635 units/mg) in CMF-PBS for 20 min.

2 mg/ml TPCK-Trypsin (Worthington 3740, 237 units/mg) in CMF-PBS for 20 min.

2 mg/ml Collagenase-2 (Worthington 4176, 150 units/mg) in D-PBS for 2 hr.

2 mg/ml Collagenase-2 in D-PBS for 3 hr.

The cells released during the last collagenase digest are separated from undigested tissue by sieving through a 100 um cell strainer (Falcon #2360). Cells are pelleted by centrifugation at 3000 rpm (Sorvall RT-6000) for 5 min.

The pellet is resuspended in culture medium (Ham's F-12+10% FBS+pen-strep), and cell number and viability are determined by trypan blue exclusion. Cells are plated in 6-well plates. Cells are allowed to attach, without disturbance, for three days, at which point the medium is changed by aspiration of the plating medium and replacement with an equal volume of culture medium. After another two to three days the medium is changed again as indicated. The day following the last medium change, the cells should have reached confluence and experimental treatments can begin. Cells are treated with 1.5 ml/well low glucose DMEM+50 μg/ml ascorbic acid for 42 hours. Replace media with 1 ml/well low glucose DMEM+50 μg/ml ascorbic acid+60 μCi/ml Na₂³⁵SO₄ for 6 hours. Conditioned media was dialyzed with a 12-14 kDa molecular weight cut-off against dH₂O. Matrix was extracted with 1.25 ml/well 4 M GuHCL+10 mM CHAPS+protease inhibitors and then dialyzed against dH₂O. 25 μl of each sample was quantitated.

We claim:

1. A method of promoting bone growth in a mammal which comprises administration of a therapeutically effective amount of a pharmaceutical composition comprising a LP85 polypeptide.

2. A method of treating of bone fractures in a mammal which comprises administration of a therapeutically effective amount of a pharmaceutical composition comprising a LP85 polypeptide.

3. A method of prophylactically increasing or maintaining bone density in a mammal having a substantially normal bone density which comprises administration of a pharmaceutical composition comprising an effective amount LP85 polypeptide.

4. A method of treating osteoporosis in a mammal which comprises administration of a therapeutically effective amount of a pharmaceutical composition comprising a LP85 polypeptide.

5. A method of treating arthritis in a mammal which comprises administration of a therapeutically effective amount of a pharmaceutical composition comprising a LP85 polypeptide.

6. A method for treating sarcopenia in a mammal which comprises administration of a therapeutically effective amount of a pharmaceutical composition comprising a LP85 polypeptide.

7. A method for treating periodontal disease in a mammal which comprises administration of a therapeutically effective amount of a pharmaceutical composition comprising a LP85 polypeptide.

8. A method for preventing cartilage degradation or promoting cartilage differentiation and function in a mammal which comprises administration of a therapeutically effective amount of a pharmaceutical composition comprising a LP85 polypeptide.

9. A method for promoting wound healing in a mammal which comprises administration of a therapeutically effective amount of a pharmaceutical composition comprising a LP85 polypeptide.

10. A method for preventing neuronal degeneration or promoting neuron growth in a mammal which comprises administration of a therapeutically effective amount of a pharmaceutical composition which comprises a LP85 polypeptide.

11. The method of claims 1-10 wherein the LP85 polypeptide comprises amino acids 270 through 370 of SEQ ID NO:2.

12. The methods of claim 1-10 wherein the LP85 polypeptide comprises amino acids 250 through 370 of SEQ ID NO:2.

13. The methods of claim 1-10 wherein the LP85 polypeptide comprises amino acids 175 through 370 of SEQ ID NO:2.

14. An isolated nucleic acid molecule encoding a LP85 analog selected from the group consisting of:
a) a LP85 analog comprising a LP85 polypeptide comprising at least one amino acid substitution at or near a region susceptible to glycosylation;
b) a LP85 analog comprising a LP85 polypeptide comprising at least one amino acid substitution at or near a region susceptible to N-glycosylation;
c) a LP85 analog comprising a LP85 polypeptide comprising at least one amino acid substitution at or near a region susceptible to N-glycosylation with high-mannose structures; and
d) a LP85 analog comprising a LP85 polypeptide comprising at least one amino acid substitution at or near Asn276 wherein said analog is less susceptible to glycosylation with high-mannose structures.

15. The LP85 analog of claim 15 wherein said amino acid substitution is selected from the group consisting of:
a. Asn at position 276 as shown in SEQ ID NO:2 is replaced by a Gln;
b. Asn at position 276 as shown in SEQ ID NO:2 is replaced by a Asp;
c. Asn at position 276 as shown in SEQ ID NO:2 is replaced by a Glu;
d. Asn at position 276 as shown in SEQ ID NO:2 is replaced by a Thr;
e. Asn at position 276 as shown in SEQ ID NO:2 is replaced by a Ala;
f. Asn at position 276 as shown in SEQ ID NO:2 is replaced by a Ser;
g. Asn at position 276 as shown in SEQ ID NO:2 is replaced by a Gln;
h. Ser at position 278 as shown in SEQ ID NO: 2 is replaced by any naturally occurring amino acid other than Ser or Thr;
i. Tyr at position 277 as shown in SEQ ID NO:2 is replaced with proline; and
j. Val at position 279 as shown in SEQ ID NO:2 is replaced with proline.

16. A method of promoting bone growth in a mammal which comprises administration of a therapeutically effective amount of a pharmaceutical composition comprising the LP85 analog of claim 15.

17. A method of treating of bone fractures in a mammal which comprises administration of a therapeutically effective amount of a pharmaceutical composition comprising the LP85 analog of claim 15.

18. A method of prophylactically increasing or maintaining bone density in a mammal having a substantially normal bone density which comprises administration of a pharmaceutical composition comprising an effective amount the LP85 analog of claim 15.

19. A method of treating osteoporosis in a mammal which comprises administration of a therapeutically effective amount of a pharmaceutical composition comprising the LP85 analog of claim 15.

20. A method of treating arthritis in a mammal which comprises administration of a therapeutically effective amount of a pharmaceutical composition comprising the LP85 analog of claim 15.

21. A method for treating sarcopenia in a mammal which comprises administration of a therapeutically effective amount of a pharmaceutical composition comprising the LP85 analog of claim 15.

22. A method for treating periodontal disease in a mammal which comprises administration of a therapeutically effective amount of a pharmaceutical composition comprising the LP85 analog of claim 15.

23. A method for preventing cartilage degradation or promoting cartilage differentiation and function in a mammal which comprises administration of a therapeutically effective amount of a pharmaceutical composition comprising the LP85 analog of claim 15.

24. A method for promoting wound healing in a mammal which comprises administration of a therapeutically effective amount of a pharmaceutical composition comprising the LP85 analog of claim 15.

25. A method for preventing neuronal degeneration or promoting neuron growth in a mammal which comprises administration of a therapeutically effective amount of a pharmaceutical composition comprising the LP85 analog of claim 15.

26. A pharmaceutical composition comprising as an active ingredient the LP85 analog of claim 15 associated with one or more pharmaceutically acceptable carriers, excipients, or diluents thereof.

27. A fusion protein comprising the LP85 analog of claim 15.

28. A nucleic acid encoding the LP85 analog of claim 15.

29. A method for producing a LP85 analog resistant to clearing in vivo comprising the step of altering the amino acid sequence in the region at and/or between positions 275 through 279 of SEQ ID NO:2.

30. A method for producing a clearing resistant LP85 analog, as in claim 31, wherein said step of altering the amino acid sequence in the region at and/or between positions 275 through 279 of SEQ ID NO:2 is selected from the group consisting of:
a. replacing Asn at position 276 as shown in SEQ ID NO:2 with a Gln;
b. replacing Asn at position 276 as shown in SEQ ID NO:2 with a Asp;
c. replacing Asn at position 276 as shown in SEQ ID NO: 2 with a Glu;
d. replacing Asn at position 276 as shown in SEQ ID NO:2 with a Thr;
e. replacing Asn at position 276 as shown in SEQ ID NO:2 with a Ala;
f. replacing Asn at position 276 as shown in SEQ ID NO:2 with a Ser;
g. replacing Asn at position 276 as shown in SEQ ID NO:2 with a Gln;
h. replacing Ser at position 278 as shown in SEQ ID NO:2 with any naturally occurring amino acid other than Ser or Thr; and
i. replacing Tyr at position 277 as shown in SEQ ID NO:2 with proline.
j. replacing Val at position 279 as shown in SEQ ID NO:2 with proline.

31. A vector comprising a nucleic acid of claim 28.

32. A recombinant host cell comprising a vector of claim 31.

33. Use of a LP85 polypeptide for the manufacture of a medicament to promote bone growth in a mammal.

34. Use of a LP85 polypeptide for the manufacture of a medicament to prevent bone loss in a mammal.

35. Use of a LP85 polypeptide for the manufacture of a medicament to treat or prevent a disease, condition, or disorder in a mammal selected from the group consisting of:
a. bone fractures;
b. osteoporosis;
c. arthritis;
d. sarcopenia;
e. periodontal;
f. cartilage degradation or trauma;
g. wounds;
h. tissue atrophy; and
i. neuronal degeneration or trauma.

36. The use of the LP85 analog of claim 14 or 15 for the manufacture of a medicament to promote bone growth in a mammal.

37. The use of the LP85 analog of claim 14 or 15 for the manufacture of a medicament to prevent bone loss in a mammal.

38. The use of a LP85 polypeptide for the manufacture of a medicament to treat or prevent a disease, condition, or disorder in a mammal selected from the group consisting of:
a. bone fractures;
b. osteoporosis;
c. arthritis;
d. sarcopenia;
e. periodontal;
f. cartilage degradation or trauma;
g. wounds;
h. tissue atrophy; and
i. neuronal degeneration or trauma.

39. The use of the LP85 analog of claim 14 or 15 for the manufacture of a medicament to treat or prevent a disease, condition, or disorder in a mammal selected from the group consisting of:
a. bone fractures;
b. osteoporosis;
c. arthritis;
d. sarcopenia;
e. periodontal;
f. cartilage degradation or trauma;
g. wounds;
h. tissue atrophy; and
i. neuronal degeneration or trauma.

40. A LP85 analog having one or more amino acid substitutions which destroy the Asn-X-Ser tripeptidyl sequence that starts at an amino acid position 276 to 278 of the amino acid sequence of native LP85 as shown in SEQ ID NO:2, wherein X is any amino acid except proline, such that glycosylation at Asn276 can not occur.
